(19) United States
(12) Patent Application Publication
(10) Pub. No.: US 2025/0032001 A1
(43) Pub. Date: Jan. 30, 2025

(54) LOWER LIMB MUSCLE POWER ESTIMATION DEVICE, LOWER LIMB MUSCLE POWER ESTIMATION SYSTEM, LOWER LIMB MUSCLE POWER ESTIMATION METHOD, AND RECORDING MEDIUM

(71) Applicant: NEC Corporation, Minato-ku, Tokyo (JP)

(72) Inventors: Chenhui HUANG, Tokyo (JP); Fumiyuki NIHEY, Tokyo (JP); Zhenwei WANG, Tokyo (JP); Hiroshi KAJITANI, Tokyo (JP); Yoshitaka NOZAKI, Tokyo (JP); Kenichiro FUKUSHI, Tokyo (JP)

(73) Assignee: NEC Corporation, Minato-ku, Tokyo (JP)

(21) Appl. No.: 18/716,596

(22) PCT Filed: Dec. 27, 2021
(86) PCT No.: PCT/JP2021/048551

§ 371 Date: Jun. 5, 2024

(51) Int. Cl. A61B 5/11

(52) U.S. Cl. CPC A61B 5/112

(57) ABSTRACT

A lower limb muscle power estimation device that includes a data acquisition unit that acquires feature amount data including a feature amount extracted from sensor data related to foot motion of a user, the feature amount being used to estimate lower limb muscle power of the user, a storage unit that stores an estimation model for outputting a lower limb muscle power index corresponding to input of the feature amount data, an estimation unit that inputs the acquired feature amount data to the estimation model and estimates the lower limb muscle power of the user according to the lower limb muscle power index output from the estimation model, and an output unit that outputs information on the estimated lower limb muscle power of the user.

### TECHNICAL FIELD

The present disclosure relates to a lower limb muscle power estimation device and the like that estimate lower limb muscle power using sensor data related to foot motion.

### BACKGROUND ART

As interest in healthcare grows, services that provide information corresponding to features included in walking patterns (also referred to as gait) have attracted attention. For example, a technique has been developed to analyze a gait based on sensor data measured by a sensor mounted on footwear such as a shoe. In the time-series data in the sensor data, a feature of a gait event (also referred to as a walking event) related to physical conditions appears.

PTL 1 discloses an information processing device that extracts a feature amount used for individual identification from motion information of a foot. The device of PTL 1 acquires motion information of a foot measured by a motion measurement device provided on a user's foot. The device of PTL 1 extracts a feature amount used for the user's identification from time-series data of one gait cycle included in the motion information.

PTL 2 discloses a method for proposing an exercise to improve motor function according to the walking ability of a subject. In the method of PTL 2, the walking ability of the subject is obtained from the subject walking a predetermined distance. In the method of PTL 2, the fall risk is determined from the value of the walking ability. In the method of PTL 2, an exercise menu for preventing falls and improving motor function is proposed in accordance with the walking ability and the fall risk. In the method of PTL 2, the proposed exercise menu is displayed, and the walking ability and the fall risk are also displayed.

Sit-to-stand motions are important motions in living daily life. The sit-to-stand motions are related to lower limb muscle power. Lower limb muscle power is an important index for evaluating frailty and fall risk.

NPL 1 reports a verification example of muscles related to performance in 30-second chair-stand (CS-30) test for measuring the number of chair-sit-to-stand motions in 30 seconds. NPL 1 reports a result in which the performance in the CS-30 test correlates with the muscle power of the iliopsoas, gluteus maximus, hamstrings, quadriceps femoris.

NPL 2 reports a verification example of muscles related to the performance in the CS-30 test in young women. NPL 2 reports a result suggesting that the performance in the CS-30 test is related to the muscle power of the gluteus maximus, which is the main moving muscle of the hip joint extension, and the posterior muscle of the lower limb, mainly including the hamstrings of the knee joint flexors.

NPL 3 reports the sequence of muscle contractions of lower limb muscle in a standing motion. NPL 3 reports that in the motion of freely standing up from a chair by a healthy adult, the sequence of muscle contractions of the tibialis anterior, gastrocnemius, quadriceps femoris, and biceps femoris generally falls into three patterns.

### CITATION LIST

### Patent Literature

PTL 1: WO 2020/240751 A1
PTL 2:2008-229266 A

### Non Patent Literature

NPL 1: Yusuke Chigira, “What are Lower Limb Muscles that Affect the Standing Sitting Operation Capability?”, Second Japan Training and Instruction Society Convention, poster presentation 01, 2013.
NPL 2: Chiaki Yagura, et al., “Relationship between 30-second chair-stand test and lower limb muscle function in young women”, Physical Therapy Supplement Vol. 30 Suppl. No. 2, (The Journal of the 38th Japan Physical Therapy Association) pp. 143, 2003.
NPL 3: Koji Kijima, et al., “Sequence of muscle contractions of lower limb muscles in standing motion”, Physical Therapy Supplement Vol. 31 Suppl. No. 2, (The Journal of the 39th Japan Physical Therapy Association) pp. A0245, 2004.

### SUMMARY OF INVENTION

### Technical Problem

In the method of PTL 1, individual identification is performed using a feature amount extracted from motion information of the foot measured by the motion measurement device provided on the user's foot. PTL 1 does not disclose estimating lower limb muscle power using a walking feature amount of a feature site extracted from data acquired from a sensor fitted to footwear.

In the method of PTL 2, an estimated index related to the walking ability of the subject is calculated according to acceleration measured by an accelerometer fitted to the waist of the subject. In the method of PTL 2, walking ability such as walking speed, stride, knee extension force, and back bending force is estimated according to the calculated estimated index. In the method of PTL 2, the walking ability corresponding to the motion of the waist is estimated, but the lower limb muscle power according to the foot motion cannot be verified.

By evaluating the chair-sit-to-stand as in NPL 1 to 3, lower limb muscle power can be evaluated. However, NPL 1 to 3 does not disclose a method for evaluating lower limb muscle power in daily life.

An object of the present disclosure is to provide a lower limb muscle power estimation device and the like capable of appropriately estimating lower limb muscle power in daily life.

### Solution to Problem

A lower limb muscle power estimation device according to one aspect of the present disclosure includes a data acquisition unit that acquires feature amount data including a feature amount extracted from sensor data related to foot motion of a user, the feature amount being used to estimate lower limb muscle power of the user, a storage unit that stores an estimation model for outputting a lower limb muscle power index corresponding to input of the feature amount data, an estimation unit that inputs the acquired feature amount data to the estimation model and estimates the lower limb muscle power of the user according to the lower limb muscle power index output from the estimation model, and an output unit that outputs information on the estimated lower limb muscle power of the user.

In a lower limb muscle power estimation method according to one aspect of the present disclosure incudes acquiring, by a computer, feature amount data including a feature amount extracted from sensor data related to foot motion of a user, the feature amount being used to estimate lower limb muscle power of the user, storing the acquired feature amount data to an estimation model for outputting a lower limb muscle power index corresponding to input of the feature amount data, estimating the lower limb muscle power of the user according to the lower limb muscle power index output from the estimation model, and outputting information on the estimated lower limb muscle power of the user.

A program according to one aspect of the present disclosure causes a computer to execute acquiring feature amount data that includes a feature amount extracted from sensor data related to foot motion of a user, the feature amount being used to estimate lower limb muscle power of the user, inputting the acquired feature amount data to an estimation model for outputting a lower limb muscle power index corresponding to input of the feature amount data, estimating the lower limb muscle power of the user according to the lower limb muscle power index output from the estimation model, and outputting information on the estimated lower limb muscle power of the user.

### Advantageous Effects of Invention

According to the present disclosure, it is possible to provide a lower limb muscle power estimation device and the like capable of appropriately estimating lower limb muscle power in daily life.

### BRIEF DESCRIPTION OF DRAWINGS

FIG. 1 is a block diagram illustrating an example of a configuration of a lower limb muscle power estimation system according to a first example embodiment.

FIG. 2 is a block diagram illustrating an example of a configuration of a gait measurement device included in the lower limb muscle power estimation system according to the first example embodiment.

FIG. 3 is a conceptual diagram illustrating a placement example of the gait measurement device according to the first example embodiment.

FIG. 4 is a conceptual diagram for explaining an example of a relationship between a local coordinate system and a world coordinate system set in the gait measurement device according to the first example embodiment.

FIG. 5 is a conceptual diagram for explaining a human body surface used in the description related to the gait measurement device according to the first example embodiment.

FIG. 6 is a conceptual diagram for explaining a gait cycle used in the description related to the gait measurement device according to the first example embodiment.

FIG. 7 is a graph for explaining an example of time-series data in sensor data measured by the gait measurement device according to the first example embodiment.

FIG. 8 is a diagram for explaining an example of normalization of gait waveform data extracted from the time-series data in the sensor data measured by the gait measurement device according to the first example embodiment.

FIG. 9 is a conceptual diagram for explaining an example of a gait phase cluster from which a feature amount data generation unit of the gait measurement device according to the first example embodiment extracts a feature amount.

FIG. 10 is a block diagram illustrating an example of a configuration of a lower limb muscle power estimation device included in the lower limb muscle power estimation system according to the first example embodiment.

FIG. 11 is a conceptual diagram for explaining a five-time chair-stand test for evaluating lower limb muscle power to be estimated by the lower limb muscle power estimation system according to the first example embodiment.

FIG. 12 is a table on specific examples of feature amounts extracted by the gait measurement device included in the lower limb muscle power estimation system according to the first example embodiment in order to estimate five-time sit-to-stand time.

FIG. 13 is a graph illustrating a correlation between a feature amount F1 extracted by the gait measurement device included in the lower limb muscle power estimation system according to the first example embodiment and a measured five-time sit-to-stand time.

FIG. 14 is a graph illustrating a correlation between a feature amount F2 extracted by the gait measurement device included in the lower limb muscle power estimation system according to the first example embodiment and a measured five-time sit-to-stand time.

FIG. 15 is a graph illustrating a correlation between a feature amount F3 extracted by the gait measurement device included in the lower limb muscle power estimation system according to the first example embodiment and a measured five-time sit-to-stand time.

FIG. 16 is a graph illustrating a correlation between a feature amount F4 extracted by the gait measurement device included in the lower limb muscle power estimation system according to the first example embodiment and a measured five-time sit-to-stand time.

FIG. 17 is a block diagram illustrating an example of estimation of the five-time sit-to-stand time (lower limb muscle power index) by the lower limb muscle power estimation device included in the lower limb muscle power estimation system according to the first example embodiment.

FIG. 18 is a graph illustrating a correlation between an estimated value of the five-time sit-to-stand time estimated using an estimation model generated by machine learning with gender, age, height, weight, and walking speed as explanatory variables and a measured value of the five-time sit-to-stand time.

FIG. 19 is a graph illustrating a correlation between an estimated value of the five-time sit-to-stand time estimated by the lower limb muscle power estimation device included in the lower limb muscle power estimation system according to the first example embodiment and a measured value of the five-time sit-to-stand time.

FIG. 20 is a flowchart for explaining an example of the operation of the gait measurement device included in the lower limb muscle power estimation system according to the first example embodiment.

FIG. 21 is a flowchart for explaining an example of the operation of the lower limb muscle power estimation device included in the lower limb muscle power estimation system according to the first example embodiment.

FIG. 22 is a conceptual diagram for explaining an application example of the lower limb muscle power estimation system according to the first example embodiment.

FIG. 23 is a block diagram illustrating an example of a configuration of a machine learning system according to a second example embodiment.

FIG. 24 is a block diagram illustrating an example of a configuration of a machine learning device included in a machine learning system according to the second example embodiment.

FIG. 25 is a conceptual diagram for explaining an example of machine learning by a machine learning device included in a machine learning system according to the second example embodiment.

FIG. 26 is a block diagram illustrating an example of a configuration of a lower limb muscle power estimation device according to a third example embodiment.

FIG. 27 is a block diagram illustrating an example of a hardware configuration that executes control and processing according to each example embodiment.

### EXAMPLE EMBODIMENT

Hereinafter, embodiments of the present invention will be described with Literature to the drawings. However, the example embodiments described below have technically preferable limitations for carrying out the present invention, but the scope of the invention is not limited to the following. In all the drawings used in the following description of the example embodiments, the same Literature numerals are provided to similar portions unless there is a particular reason for not doing so. In the following example embodiments, repeated description of similar configurations and operations may be omitted.

### First Example Embodiment

First, a lower limb muscle power estimation system according to a first example embodiment will be described with Literature to the drawings. The lower limb muscle power estimation system of the present example embodiment measures sensor data related to foot motion according to gait of a user. The lower limb muscle power estimation system of the present example embodiment estimates the user's lower limb muscle power, using the measured sensor data.

In the present example embodiment, as the lower limb muscle power, an example of estimating performance in a five-time chair-stand test in which the chair-stand is repeated five times will be described. Hereinafter, the five-time chair-stand test is also referred to as sit-to-stand-5 (SS-5) test. In the present example embodiment, the performance in the SS-5 test is evaluated by the time for repeating the chair-sit-to-stand five times (also referred to as five-time sit-to-stand time). The five-time sit-to-stand time is a performance value of the SS-5 test. The shorter the five-time sit-to-stand time, the higher the performance in the SS-5 test. The method of the present example embodiment can also be applied to performance in sit-to-stand tests other than the SS-5 test. For example, the method of the present example embodiment can also be applied to performance in a 30-second chair-stand (CS-30) test for measuring the number of chair-sit-to-stand motions in 30 seconds.

### (Configuration)

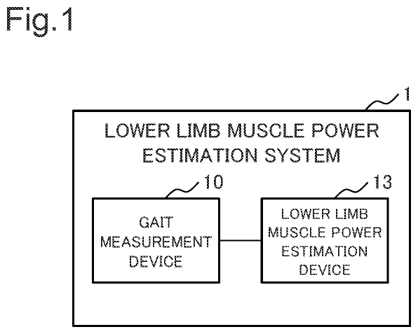

FIG. 1 is a block diagram illustrating an example of a configuration of a lower limb muscle power estimation system 1 according to the present example embodiment. The lower limb muscle power estimation system 1 includes a gait measurement device 10 and a lower limb muscle power estimation device 13. In the present example embodiment, an example in which the gait measurement device 10 and the lower limb muscle power estimation device 13 are configured as separate hardware will be described. For example, the gait measurement device 10 is fitted to footwear or the like of a subject (user) who is an estimation target for lower limb muscle power. For example, the function of the lower limb muscle power estimation device 13 is installed in a mobile terminal carried by a subject (user). Hereinafter, the configurations of the gait measurement device 10 and the lower limb muscle power estimation device 13 will be described individually.

### [Gait Measurement Device]

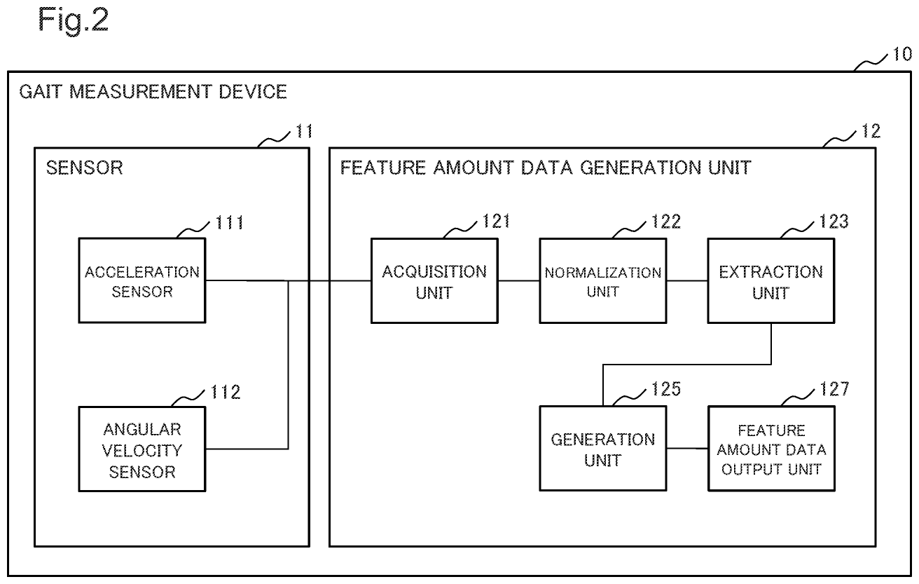

FIG. 2 is a block diagram illustrating an example of the configuration of the gait measurement device 10. The gait measurement device 10 includes a sensor 11 and a feature amount data generation unit 12. In the present example embodiment, an example in which the sensor 11 and the feature amount data generation unit 12 are integrated will be described. The sensor 11 and the feature amount data generation unit 12 may be provided as separate devices.

As illustrated in FIG. 2, the sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include a sensor other than the acceleration sensor 111 and the angular velocity sensor 112. A sensor other than the acceleration sensor 111 and the angular velocity sensor 112 that can be included in the sensor 11 will not be described.

The acceleration sensor 111 is a sensor that measures accelerations (also referred to as spatial accelerations) in the three axial directions. The acceleration sensor 111 measures acceleration (also referred to as spatial acceleration) as a physical quantity related to foot motion. The acceleration sensor 111 outputs the measured acceleration to the feature amount data generation unit 12. For example, a sensor of a piezoelectric type, a piezoresistive type, a capacitance type, or the like can be used as the acceleration sensor 111. The sensor used as the acceleration sensor 111 does not limit its measurement method as long as the sensor can measure acceleration.

The angular velocity sensor 112 is a sensor that measures angular velocities (also referred to as a spatial angular velocity) around three axes. The angular velocity sensor 112 measures angular velocity (also referred to as spatial angular velocity) as a physical quantity related to the foot motion. The angular velocity sensor 112 outputs the measured angular velocity to the feature amount data generation unit 12. For example, a sensor of a vibration type, a capacitance type, or the like can be used as the angular velocity sensor 112. The sensor used as the angular velocity sensor 112 does not limit its measurement method as long as the sensor can measure the angular velocity.

The sensor 11 is achieved by, for example, an inertial measurement device that measures acceleration and angular velocity. An example of the inertial measurement unit is an inertial measurement unit (IMU). The IMU includes an acceleration sensor 111 that measures accelerations in the three axial directions and an angular velocity sensor 112 that measures angular velocities around the three axes. The sensor 11 may be achieved by an inertial measurement device such as a vertical gyro (VG) or an attitude and heading reference system (AHRS). The sensor 11 may be achieved by the global positioning system/inertial navigation system (GPS/INS). The sensor 11 may be achieved by a device other than the inertial measurement device as long as a physical quantity related to the foot motion can be measured.

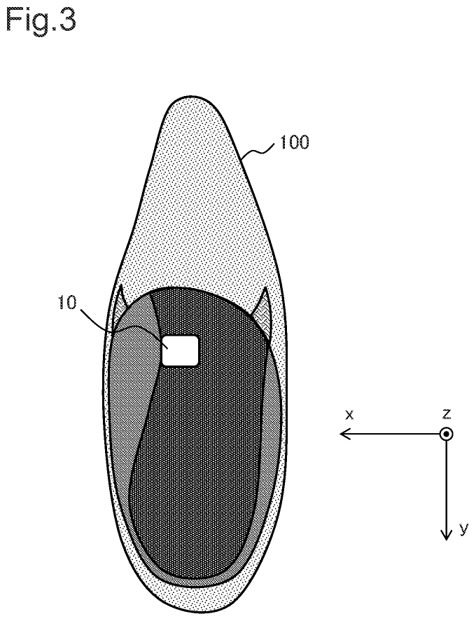

FIG. 3 is a conceptual diagram illustrating an example in which the gait measurement device 10 is placed in the shoe 100 of the right foot. In the example of FIG. 3, the gait measurement device 10 is fitted to a position corresponding to the back side of the foot arch. For example, the gait measurement device 10 is placed on an insole inserted into the shoe 100. For example, the gait measurement device 10 may be placed on the bottom surface of the shoe 100. For example, the gait measurement device 10 may be embedded in the body of the shoe 100. The gait measurement device 10 may be detachable from the shoe 100 or may not be detachable from the shoe 100. The gait measurement device 10 may be fitted to a position other than the back side of the foot arch as long as sensor data related to the foot motion can be measured. The gait measurement device 10 may be fitted to a sock worn by the user or a decorative article such as an anklet worn by the user. The gait measurement device 10 may be directly attached to the foot or may be embedded in the foot. FIG. 3 illustrates an example in which the gait measurement device 10 is fitted to a right shoe 100. The gait measurement device 10 may be fitted to both shoes 100.

In the example of FIG. 3, a local coordinate system including the x-axis in the left-right direction, the y-axis in the front-back direction, and the z-axis in the up-down direction is set with Literature to the gait measurement device 10 (sensor 11). The x-axis is positive toward the left, the y-axis is positive toward the back, and the z-axis is positive upward. The orientation of the axes set on the sensor 11 may be the same for the left and right feet, or may be different for the left and right feet. For example, in a case where the sensors 11 produced with the same specifications are placed in the left and right shoes 100, the vertical orientation (the orientation in the Z-axis) of the sensors 11 placed in the left and right shoes 100 are the same. In this case, the three axes of the local coordinate system set in the sensor data derived from the left foot and the three axes of the local coordinate system set in the sensor data derived from the right foot are the same on the left and right.

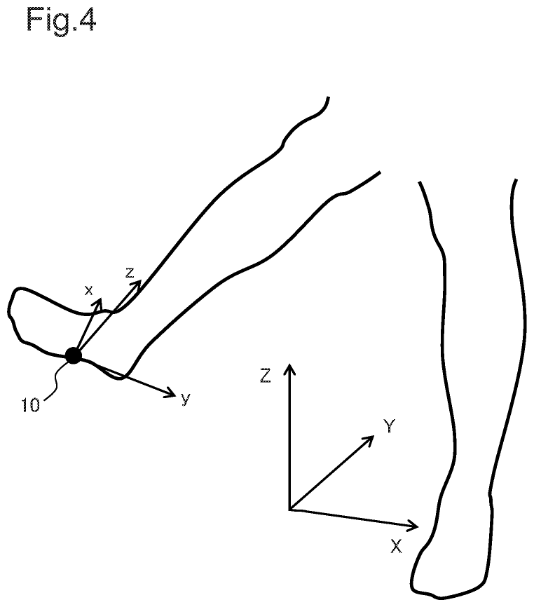

FIG. 4 is a conceptual diagram for explaining a local coordinate system (x-axis, y-axis, z-axis) set in the gait measurement device 10 (sensor 11) fitted to the back side of the foot arch and a world coordinate system (X-axis, Y-axis, Z-axis) set with respect to the ground. In the world coordinate system (X-axis, Y-axis, Z-axis), in a state where the user facing the traveling direction is upright, the direction of the use's side is set to the X-axis direction (positive toward the left), the direction of the user's back is set to the Y-axis direction (positive toward the back), and the gravity direction is set to the Z-axis direction (positive upward). The example of FIG. 4 conceptually illustrates the relationship between the local coordinate system (x-axis, y-axis, z-axis) and the world coordinate system (X-axis, Y-axis, Z-axis), and does not accurately illustrate the relationship between the local coordinate system and the world coordinate system that varies depending on the user's gait.

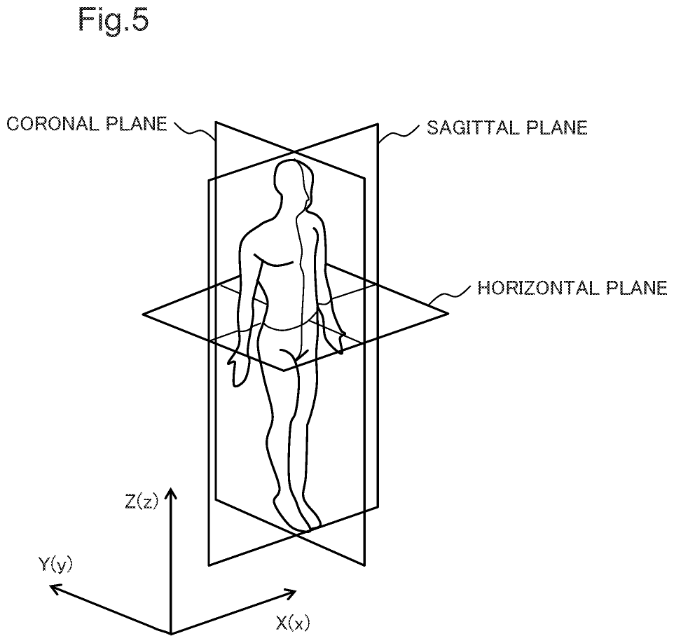

FIG. 5 is a conceptual diagram for explaining planes set for the human body (also referred to as a human body planes). In the present example embodiment, a sagittal plane dividing the body into left and right, a coronal plane dividing the body into front and rear, and a horizontal plane dividing the body horizontally are defined. As illustrated in FIG. 5, the world coordinate system and the local coordinate system coincide with each other in a state where the center line of the foot is oriented in the traveling direction. In the present example embodiment, rotation in the sagittal plane with the x-axis as the rotation axis is defined as roll, rotation in the coronal plane with the y-axis as the rotation axis is defined as pitch, and rotation in the horizontal plane with the z-axis as the rotation axis is defined as yaw. A rotation angle in a sagittal plane with the x-axis as a rotation axis is defined as a roll angle, a rotation angle in a coronal plane with the y-axis as a rotation axis is defined as a pitch angle, and a rotation angle in a horizontal plane with the z-axis as a rotation axis is defined as a yaw angle.

As illustrated in FIG. 2, the feature amount data generation unit 12 (also referred to as a feature amount data generation device) includes an acquisition unit 121, a normalization unit 122, an extraction unit 123, a generation unit 125, and a feature amount data output unit 127. For example, the feature amount data generation unit 12 is achieved by a microcomputer or a microcontroller that performs overall control and data processing of the gait measurement device 10. For example, the feature amount data generation unit 12 includes a central processing unit (CPU), a random-access memory (RAM), a read-only memory (ROM), a flash memory, and the like. The feature amount data generation unit 12 controls the acceleration sensor 111 and the angular velocity sensor 112 to measure the angular velocity and the acceleration. For example, the feature amount data generation unit 12 may be implemented on a mobile terminal (not illustrated) carried by a subject (user).

The acquisition unit 121 acquires accelerations in the three axial directions from the acceleration sensor 111. The acquisition unit 121 acquires angular velocities around three axes from the angular velocity sensor 112. For example, the acquisition unit 121 performs analog-to-digital conversion (AD conversion) on the acquired physical quantities (analog data) such as angular velocity and acceleration. The physical quantity (analog data) measured by each of the acceleration sensor 111 and the angular velocity sensor 112 may be converted into digital data in each of the acceleration sensor 111 and the angular velocity sensor 112. The acquisition unit 121 outputs the converted digital data (also referred to as sensor data) to the normalization unit 122. The acquisition unit 121 may be configured to store the sensor data in a storage unit (not illustrated). The sensor data includes at least acceleration data converted into digital data and angular velocity data converted into digital data. The acceleration data includes acceleration vectors in the three axial directions. The angular velocity data includes angular velocity vectors around three axes. Each of the acceleration data and the angular velocity data is associated with the acquisition time of the data. The acquisition unit 121 may add correction, such as a mounting error, temperature correction, and linearity correction, to the acceleration data and the angular velocity data.

The normalization unit 122 acquires sensor data from the acquisition unit 121. The normalization unit 122 extracts time-series data for one gait cycle (also referred to as gait waveform data) from the time-series data of the accelerations in the three axial directions and the angular velocities around the three axes included in the sensor data. The normalization unit 122 normalizes the time of the extracted gait waveform data for one gait cycle to a gait cycle of 0 to 100% (percent) (also referred to as first normalization). The timing included in the gait cycle of 0 to 100%, such as 1% or 10%, is also referred to as a gait phase. The normalization unit 122 normalizes the gait waveform data for one gait cycle subjected to the first normalization so that the stance phase is 60% and the swing phase is 40% (also referred to as second normalization). The stance phase is a period in which at least a part of the back side of the foot is in contact with the ground. The swinging phase is a period in which the back side of the foot is away from the ground. When the gait waveform data is subjected to the second normalization, it is possible to suppress the deviation of the gait phase from which the feature amount is extracted from fluctuating due to the influence of disturbance.

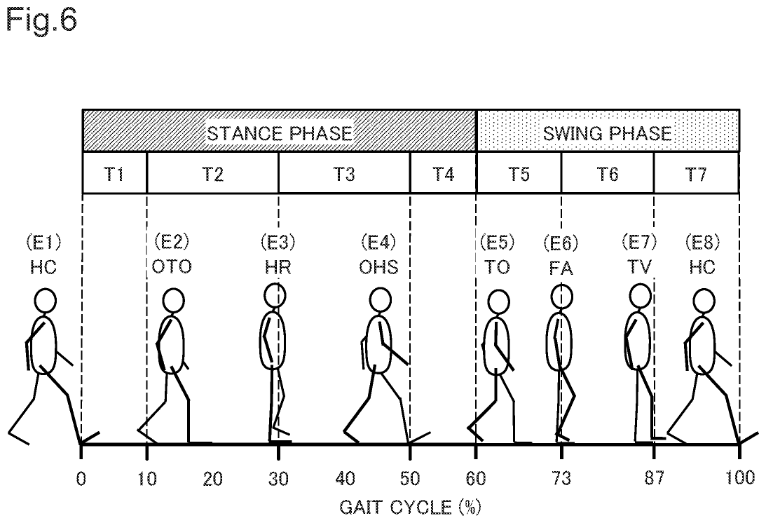

FIG. 6 is a conceptual diagram for explaining one gait cycle with the right foot as a Literature. One gait cycle based on the left foot is similar to that of the right foot. The horizontal axis of FIG. 6 is one gait cycle of the right foot with a time point at which the heel of the right foot lands on the ground as a start point and a time point at which the heel of the right foot next lands on the ground as an end point. The horizontal axis in FIG. 6 has been subjected to the first normalization with one gait cycle as 100%. In the horizontal axis of FIG. 6, the second normalization has been performed so that the stance phase is 60% and the swinging phase is 40%. The one gait cycle of one foot is roughly divided into a stance phase in which at least a part of the back side of the foot is in contact with the ground and a swinging phase in which the back side of the foot is separated from the ground. The stance phase is further subdivided into a load response period T1, a mid-stance period T2, a terminal stance period T3, and a pre-swing period T4. The swing phase is further subdivided into a swing initial stage T5, a mid-swing period T6, and a terminal swing period T7. FIG. 6 is an example, and does not limit the periods constituting one gait cycle, the names of these periods, and the like.

As illustrated in FIG. 6, in walking, a plurality of events (also referred to as walking events) occur. E1 represents an event (heel contact) in which the heel of the right foot comes into contact with the ground (HC) E2 represents an event (opposite toe-off) in which the toe of the left foot is separated from the ground with the sole of the right foot in contact with the ground (OTO). E3 represents an event (heel rise) in which the heel of the right foot lifts with the sole of the right foot in contact with the ground (HR). E4 is an event (opposite heel strike) in which the heel of the left foot is grounded (OHS). E5 represents an event (toe-off) in which the toe of the right foot is separated from the ground in a state where the sole of the left foot is grounded (TO). E6 represents an event (foot adjacent) in which the left foot and the right foot cross with the sole of the left foot in contact with the ground (FA). E7 represents an event (tibia vertical) in which the tibia of the right foot is approximately perpendicular to the ground (tibia vertical) with the sole of the left foot in contact (TV). E8 represents an event (heel contact) in which the heel of the right foot comes into contact with the ground (HC). E8 corresponds to the end point of the gait cycle starting from E1 and corresponds to the start point of the next gait cycle. FIG. 6 is an example, and does not limit events that occur in walking or names of these events.

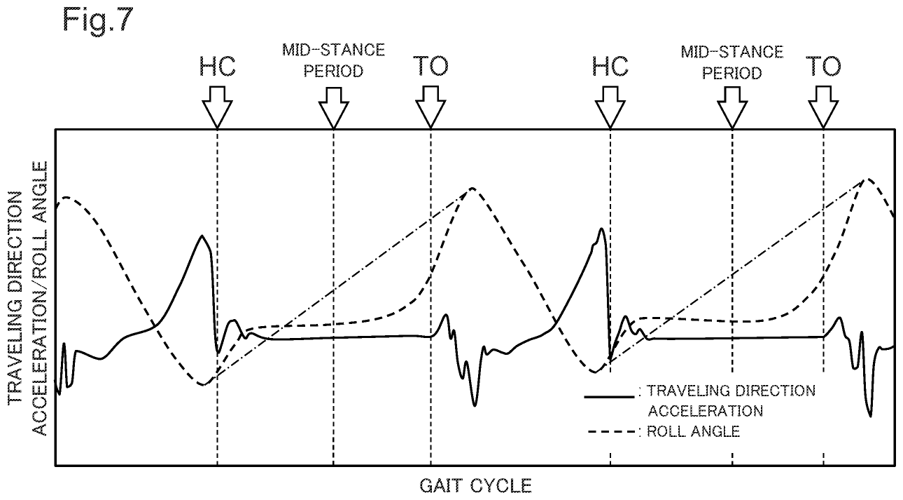

FIG. 7 is a diagram for explaining an example of detecting a heel contact HC and a toe-off TO from the time-series data (solid line) of the traveling-direction acceleration (Y-direction acceleration). The timing for the heel contact HC is the timing for the minimum peak immediately after the maximum peak appearing in the time-series data of the traveling-direction acceleration (Y-direction acceleration). The maximum peak serving as the mark of the timing for the heel contact HC corresponds to the maximum peak of the gait waveform data for one gait cycle. A section between consecutive heel contacts HC is one gait cycle. The timing for the toe-off TO is the rising timing for the maximum peak appearing after the period of the stance phase in which the fluctuation does not appear in the time-series data of the traveling-direction acceleration (Y-direction acceleration). FIG. 7 also illustrates the time-series data (broken line) of the roll angle (angular velocity around the X-axis). The timing at the midpoint between the timing at which the roll angle is minimum and the timing at which the roll angle is maximum corresponds to the mid-stance period. For example, parameters (also referred to as gait parameters) such as walking speed, stride, minute, medial/lateral rotation, and plantarflexion/dorsiflexion can be obtained with Literature to the mid-stance period.

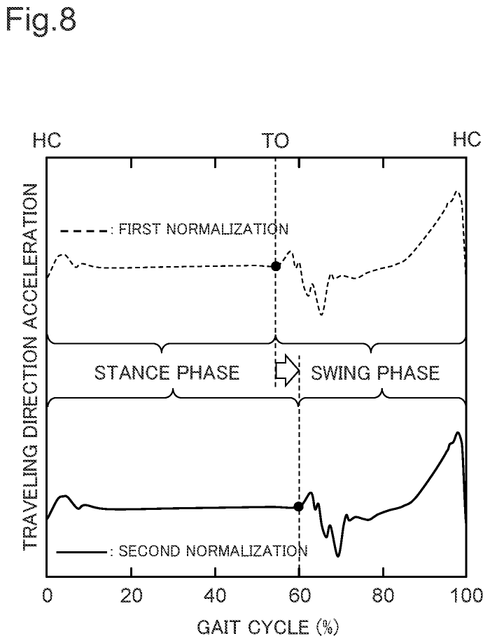

FIG. 8 is a diagram for explaining an example of the gait waveform data normalized by the normalization unit 122. The normalization unit 122 detects the heel contact HC and the toe-off TO from the time-series data of the traveling-direction acceleration (Y-direction acceleration). The normalization unit 122 extracts a section between consecutive heel contacts HC as gait waveform data for one gait cycle. The normalization unit 122 converts the horizontal axis (time axis) of the gait waveform data for one gait cycle into a gait cycle of 0 to 100% by the first normalization. In FIG. 8, the gait waveform data after the first normalization is indicated by a broken line. In the gait waveform data (broken line) after the first normalization, the timing for the toe-off TO deviates from 60%.

In the example of FIG. 8, the normalization unit 122 normalizes a section from the heel contact HC at which the gait phase is 0% to the toe-off TO subsequent to the heel contact HC to 0 to 60%. The normalization unit 122 normalizes a section from the toe-off TO to the heel contact HC in which the gait phase subsequent to the toe-off TO is 100% to 60 to 100%. As a result, the gait waveform data for one gait cycle is normalized to a section (stance phase) in which the gait cycle is 0 to 60% and a section (swinging phase) in which the gait cycle is 60 to 100%. In FIG. 8, the gait waveform data after the second normalization is indicated by a solid line. In the gait waveform data (solid line) after the second normalization, the timing for the toe-off TO coincides with 60%.

FIGS. 7 to 8 illustrate examples in which the gait waveform data for one gait cycle is extracted/normalized based on the traveling-direction acceleration (Y-direction acceleration). With respect to the acceleration/angular velocity other than the traveling-direction acceleration (Y-direction acceleration), the normalization unit 122 extracts/normalizes gait waveform data for one gait cycle in accordance with the gait cycle of the traveling-direction acceleration (Y-direction acceleration). The normalization unit 122 may generate time-series data of angles around three axes by integrating the time-series data of angular velocities around the three axes. In this case, the normalization unit 122 also extracts/normalizes the gait waveform data for one gait cycle in accordance with the gait cycle of the traveling-direction acceleration (Y-direction acceleration) with respect to the angles around the three axes.

The normalization unit 122 may extract/normalize the gait waveform data for one gait cycle based on acceleration/angular velocity other than the traveling-direction acceleration (Y-direction acceleration) (not illustrated). For example, the normalization unit 122 may detect the heel contact HC and the toe-off TO from the time-series data of the vertical-direction acceleration (Z-direction acceleration). The timing for the heel contact HC is the timing for a steep minimum peak appearing in the time-series data of the vertical-direction acceleration (Z-direction acceleration). At the timing for the steep minimum peak, the value of the vertical-direction acceleration (Z-direction acceleration) becomes substantially zero. The minimum peak serving as the mark of the timing for the heel contact HC corresponds to the minimum peak of the gait waveform data for one gait cycle. A section between consecutive heel contacts HC is one gait cycle. The timing for the toe-off TO is the timing for an inflection point in the middle of a gradual increase in the time-series data of the vertical-direction acceleration (Z-direction acceleration) after its passage through a section with a small fluctuation after the maximum peak immediately after the heel contact HC. The normalization unit 122 may extract/normalize the gait waveform data for one gait cycle based on both the traveling-direction acceleration (Y-direction acceleration) and the vertical-direction acceleration (Z-direction acceleration). The normalization unit 122 may extract/normalize the gait waveform data for one gait cycle based on acceleration, angular velocity, angle, and the like other than the traveling-direction acceleration (Y-direction acceleration) and the vertical-direction acceleration (Z-direction acceleration).

The extraction unit 123 acquires gait waveform data for one gait cycle normalized by the normalization unit 122. The extraction unit 123 extracts a feature amount to be used to estimate lower limb muscle power from gait waveform data for one gait cycle. The extraction unit 123 extracts a feature amount for each gait phase cluster from a gait phase cluster obtained by integrating temporally continuous gait phases, based on a preset condition. The gait phase cluster includes at least one gait phase. The gait phase cluster also includes a single gait phase. The gait waveform data and the gait phase from which the feature amount to be used to estimate the lower limb muscle power are extracted will be described later.

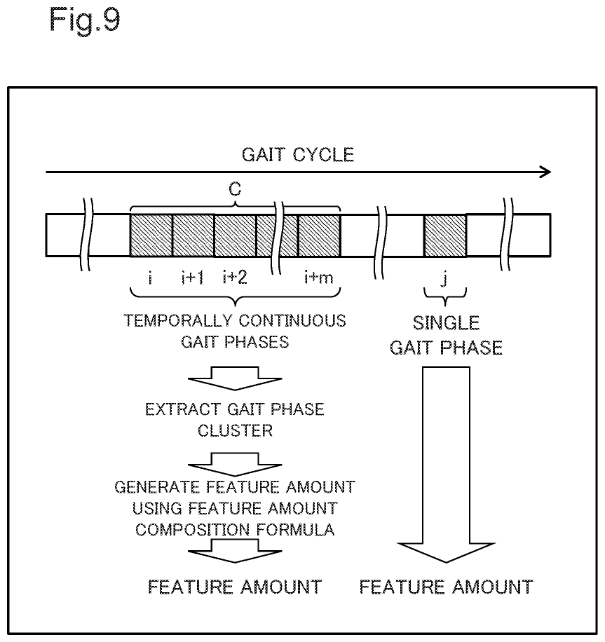

FIG. 9 is a conceptual diagram for explaining extraction of a feature amount for estimating lower limb muscle power from gait waveform data for one gait cycle. For example, the extraction unit 123 extracts temporally continuous gait phases i to i+m as a gait phase cluster C (i and m are natural numbers). The gait phase cluster C includes m gait phases (components). That is, the number of gait phases (components) (also referred to as the number of components) constituting the gait phase cluster C is m. FIG. 9 illustrates an example in which the gait phase has an integer value, but the gait phase may be subdivided into decimal places. When the gait phase is subdivided into decimal places, the number of components of the gait phase cluster C is a number corresponding to the number of data points in the section of the gait phase cluster. The extraction unit 123 extracts a feature amount from each of the gait phases i to i+m. When the gait phase cluster C is formed of a single gait phase j, the extraction unit 123 extracts a feature amount from the single gait phase j (j is a natural number).

The generation unit 125 applies a feature amount composition formula to the feature amount (first feature amount) extracted from each of the gait phases constituting the gait phase cluster to generate the feature amount (second feature amount) of the gait phase cluster. The feature amount composition formula is a preset calculation formula for generating the feature amount of the gait phase cluster. For example, the feature amount composition formula is a calculation formula related to four arithmetic operations. For example, the second feature amount calculated using the feature amount composition formula is an integral average value, an arithmetic average value, a slope, a variation, or the like of the first feature amount in each gait phase included in the gait phase cluster. For example, the generation unit 125 applies a calculation formula for calculating the inclination and variation of the first feature amount extracted from each of the gait phases constituting the gait phase cluster as the feature amount composition formula. For example, when the gait phase cluster is formed of a single gait phase, it is not possible to calculate the inclination or variation, and hence it is sufficient to use a feature amount composition formula for calculating an integral average value, an arithmetic average value, or the like.

The feature amount data output unit 127 outputs the feature amount data for each gait phase cluster generated by the generation unit 125. The feature amount data output unit 127 outputs the generated feature amount data of the gait phase cluster to the lower limb muscle power estimation device 13 using the feature amount data.

### [Lower Limb Muscle Power Estimation Device]

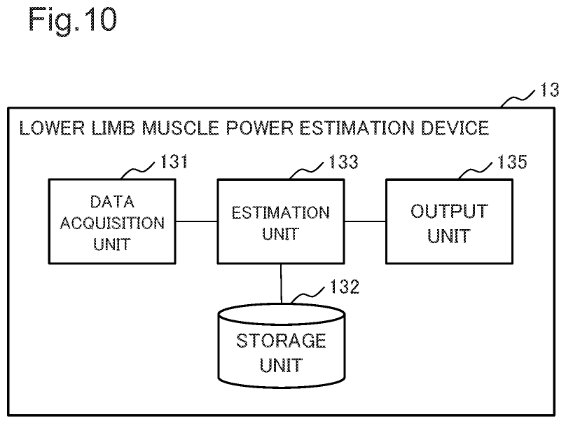

FIG. 10 is a block diagram illustrating an example of the configuration of the lower limb muscle power estimation device 13. The lower limb muscle power estimation device 13 includes a data acquisition unit 131, a storage unit 132, an estimation unit 133, and an output unit 135.

The data acquisition unit 131 acquires feature amount data from the gait measurement device 10. The data acquisition unit 131 outputs the received feature amount data to the estimation unit 133. The data acquisition unit 131 may receive the feature amount data from the gait measurement device 10 via a wire such as a cable, or may receive the feature amount data from the gait measurement device 10 via wireless communication. For example, the data acquisition unit 131 is configured to receive the feature amount data from the gait measurement device 10 via a wireless communication function (not illustrated) conforming to a standard such as Bluetooth (registered trademark) or WiFi (registered trademark). The communication function of the data acquisition unit 131 may conform to a standard other than Bluetooth (registered trademark) or WiFi (registered trademark).

The storage unit 132 stores an estimation model for estimating the five-time sit-to-stand time as the lower limb muscle power index using the feature amount data extracted from the gait waveform data. The storage unit 132 stores an estimation model that has learned the relationship between feature amount data related to the five-time sit-to-stand time for a plurality of subjects and the five-time sit-to-stand time. For example, the storage unit 132 stores an estimation model that has learned for the plurality of subjects to estimate the five-time sit-to-stand time. The five-time sit-to-stand time is affected by age. Therefore, the storage unit 132 may store an estimation model corresponding to attribute data related to age.

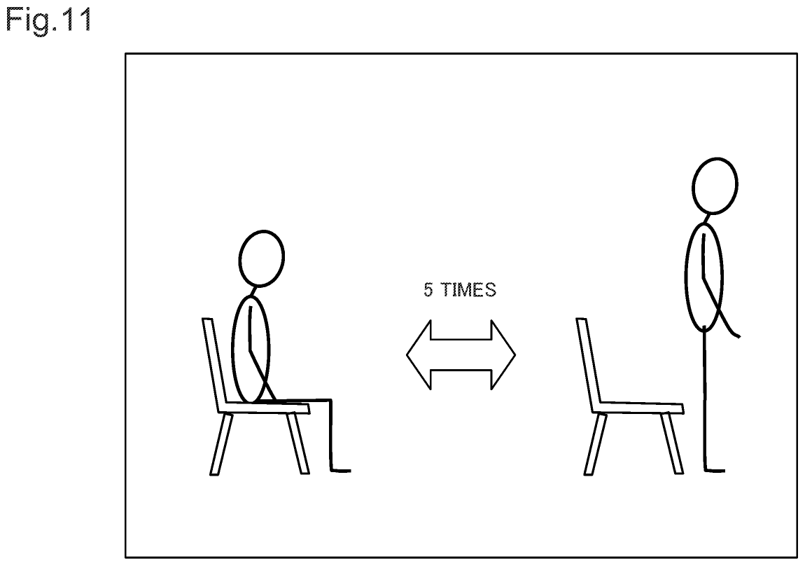

FIG. 11 is a conceptual diagram for explaining the five-time sit-to-stand test (SS-5 test). The subject repeats the motion of standing up from a seated position on a chair five times. The measurement is started from the seated position on the chair, the user repeatedly stands and sits, and the measurement ends at the time when the user stands up five times.

Lower limb muscle power can be evaluated according to the five-time sit-to-stand time. In the case of age 60 to 69, the average value of the five-time sit-to-stand time is about 11 to 12 seconds. In the case of age 70 to 89, the average value of the five-time sit-to-stand time is about 12 to 13 seconds. When it takes about 14 to 15 seconds for the five-time sit-to-stand time, the fall risk is high. The evaluation criteria of the lower limb muscle power corresponding to the five-time sit-to-stand time described herein is a guide, and may be set according to the situation.

The estimation model may be stored in the storage unit 132 at the time of factory shipment of a product, calibration before the user uses the lower limb muscle power estimation system 1, or the like. For example, an estimation model stored in a storage device such as an external server may be used. In that case, the estimation model may be configured to be used via an interface (not illustrated) connected to the storage device.

The estimation unit 133 acquires the feature amount data from the data acquisition unit 131. The estimation unit 133 executes the estimation of the five-time sit-to-stand time as lower limb muscle power using the acquired feature amount data. The estimation unit 133 inputs the feature amount data to the estimation model stored in the storage unit 132. The estimation unit 133 outputs an estimation result corresponding to the lower limb muscle power (five-time sit-to-stand time) output from the estimation model. When an estimation model, stored in an external storage device constructed in a cloud, a server, or the like, is used, the estimation unit 133 is configured to use the estimation model via an interface (not illustrated) connected to the storage device.

The output unit 135 outputs the estimation result of the lower limb muscle power obtained by the estimation unit 133. For example, the output unit 135 displays the estimation result of the lower limb muscle power on the screen of the mobile terminal of the subject (user). For example, the output unit 135 outputs the estimation result to an external system or the like that uses the estimation result. The use of the lower limb muscle power output from lower limb muscle power estimation device 13 is not particularly limited.

For example, the lower limb muscle power estimation device 13 is connected to an external system or the like constructed in a cloud or a server via a mobile terminal (not illustrated) carried by the subject (user). The mobile terminal (not illustrated) is a portable communication device. For example, the mobile terminal is a portable communication device having a communication function, such as a smartphone, a smart watch, or a mobile phone. For example, the lower limb muscle power estimation device 13 is connected to the mobile terminal via a wire such as a cable. For example, the lower limb muscle power estimation device 13 is connected to the mobile terminal via wireless communication. For example, the lower limb muscle power estimation device 13 is connected to the mobile terminal via a wireless communication function (not illustrated) conforming to a standard such as Bluetooth (registered trademark) or WiFi (registered trademark). The communication function of the lower limb muscle power estimation device 13 may conform to a standard other than Bluetooth (registered trademark) or WiFi (registered trademark). The lower limb muscle power estimation result may be used by an application installed in the mobile terminal. In that case, the mobile terminal executes processing using the estimation result using application software or the like installed in the mobile terminal.

### [Estimation of Five-Time Sit-to-Stand Time]

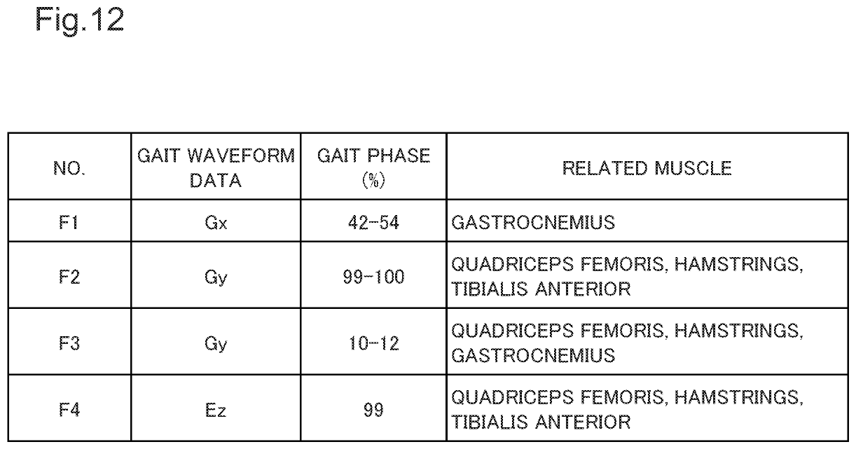

Next, the correlation between the five-time sit-to-stand time and the feature amount data will be described with Literature to a verification example. FIG. 12 is a correspondence table summarizing feature amounts to be used to estimate the five-time sit-to-stand time. The correspondence table of FIG. 12 associates the number of the feature amount, the gait waveform data from which the feature amount is extracted, the gait phase (%) from which the gait phase cluster is extracted, and the related muscles. The five-time sit-to-stand time is correlated with quadriceps femoris, hamstrings, tibialis anterior, and gastrocnemius. Therefore, feature amounts F1 to F4 extracted from the gait phase in which these features appear are used to estimate the five-time sit-to-stand time.

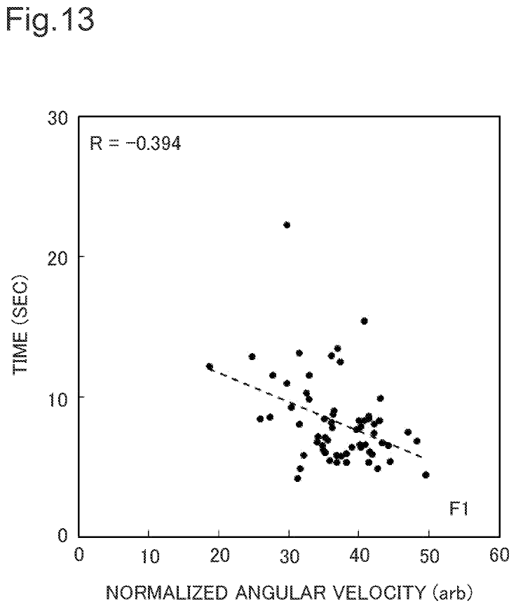

FIGS. 13 to 16 are verification results of the correlation between the five-time sit-to-stand time and the feature amount data. FIGS. 13 to 16 illustrate the results of verification performed on a total of 62 subjects including 27 males and 35 females aged 60 to 85 years. FIGS. 13 to 16 illustrate results of verifying the correlation between the estimated value estimated using the feature amount extracted according to the walking in footwear mounted with the gait measurement device 10 and the measured value (true value) of the five-time sit-to-stand time.

The feature amount F1 is extracted from a section of a 42 to 54% gait phase of gait waveform data Gx related to the time-series data of the angular velocity in the sagittal plane (around the X-axis). The 42 to 54% gait phase is a section from the terminal stance period T3 to the pre-swing period T4. The feature amount F1 mainly includes a feature related to the motion of the gastrocnemius. FIG. 13 is a verification result of the correlation between the feature amount F1 and the five-time sit-to-stand time. The horizontal axis of the graph of FIG. 13 is the normalized angular velocity. A correlation coefficient R between the feature amount F1 and the five-time sit-to-stand time was −0.394.

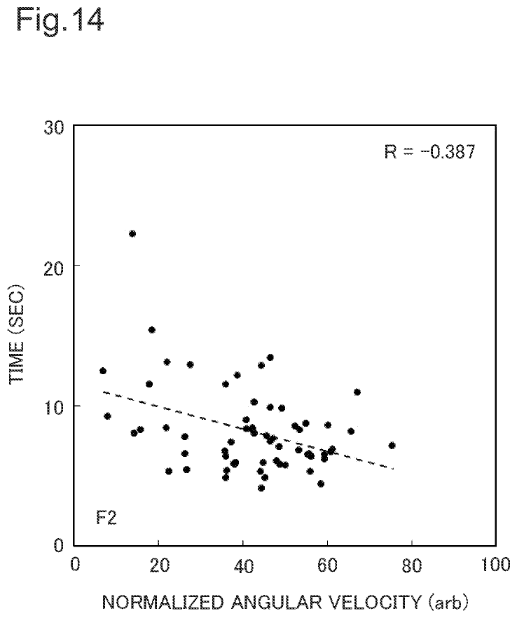

The feature amount F2 is extracted from a section of a 99 to 100% gait phase of gait waveform data Gy related to the time-series data of the angular velocity in the coronal plane (around the Y-axis). The 99 to 100% gait phase is the final stage of the terminal swing period T7. The feature amount F2 mainly includes features related to the motion of the quadriceps femoris, hamstrings, and tibialis anterior. FIG. 14 is a verification result of the correlation between the feature amount F2 and the five-time sit-to-stand time. The horizontal axis of the graph of FIG. 14 is the normalized angular velocity. The correlation coefficient R between the feature amount F2 and the five-time sit-to-stand time was −0.387.

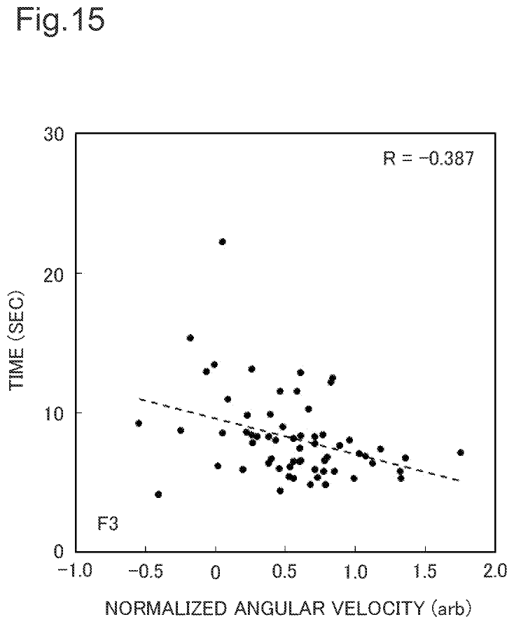

The feature amount F3 is extracted from a section of a 10 to 12% gait phase of gait waveform data Gy related to the time-series data of the angular velocity in the coronal plane (around the Y-axis). The 10 to 12% gait phase is the early stage of the mid-stance period T2. The feature amount F3 mainly includes features related to the motion of the quadriceps femoris, hamstrings, and gastrocnemius. FIG. 15 is a verification result of the correlation between the feature amount F3 and the five-time sit-to-stand time. The horizontal axis of the graph of FIG. 15 is the normalized angular velocity. The correlation coefficient R between the feature amount F4 and the five-time sit-to-stand time was −0.387.

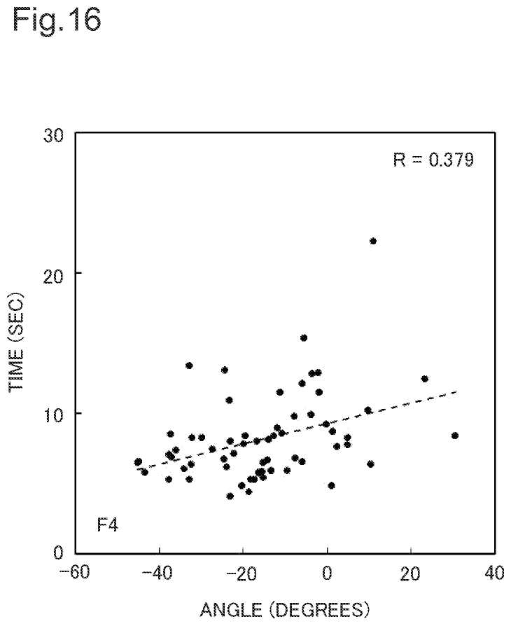

The feature amount F4 is extracted from a section of a 99% gait phase of gait waveform data Ez related to the time-series data of the angle (posture angle) in the horizontal plane (around the Z-axis). The 99% gait phase is the final stage of the terminal swing period T7. The feature amount F4 mainly includes features related to the motion of the quadriceps femoris, hamstrings, and tibialis anterior. FIG. 16 is a verification result of the correlation between the feature amount F4 and the five-time sit-to-stand time. The horizontal axis of the graph of FIG. 16 is the angle in the horizontal plane (around the Z-axis). The correlation coefficient R between the feature amount F4 and the five-time sit-to-stand time was 0.379.

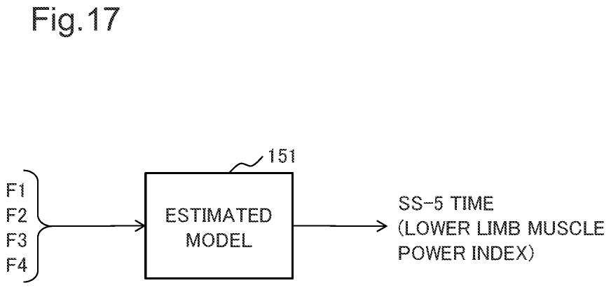

FIG. 17 is a conceptual diagram illustrating an example in which the feature amounts F1 to F4 extracted from sensor data measured along with the user's gait are input to an estimation model 151 constructed in advance for estimating five-time sit-to-stand time as lower limb muscle power. The estimation model 151 outputs the five-time sit-to-stand time, which is a lower limb muscle power index, according to the inputs of the feature amounts F1 to F4. For example, the estimation model 151 is generated by machine learning using teacher data with the feature amounts F1 to F4 to be used to estimate the five-time sit-to-stand time as explanatory variables and the five-time sit-to-stand time as an objective variable. As long as the estimation result related to the five-time sit-to-stand time, which is an index of lower limb muscle power, is output in response to the input of the feature amount data for estimating the five-time sit-to-stand time, the estimation result of the estimation model 151 is not limited. For example, the estimation model 151 may be a model that estimates the five-time sit-to-stand time using the attribute data (age) as an explanatory variable in addition to the feature amounts F1 to F4 to be used to estimate the five-time sit-to-stand time.

For example, the storage unit 132 stores an estimation model for estimating the five-time sit-to-stand time using a multiple regression prediction method. For example, the storage unit 132 stores a parameter for estimating a five-time sit-to-stand time T using the following formula

$$T = a 1 \times F 1 + a 2 \times F 2 + a 3 \times F 3 + a 4 \times F 4 + a 0 \qquad ( 1 )$$

In Formula 1 above, F1, F2, F3, and F4 are feature amounts for the respective gait phase clusters to be used to estimate the five-time sit-to-stand time illustrated in the correspondence table of FIG. 12. a1, a2, a3, and a4 are coefficients multiplied by F1, F2, F3, and F4. a0 is a constant term. For example, a0, a1, a2, a3, and a4 are stored in the storage unit 132.

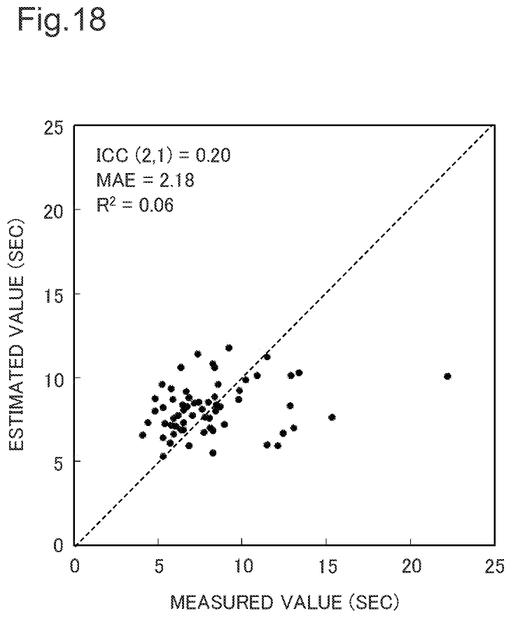

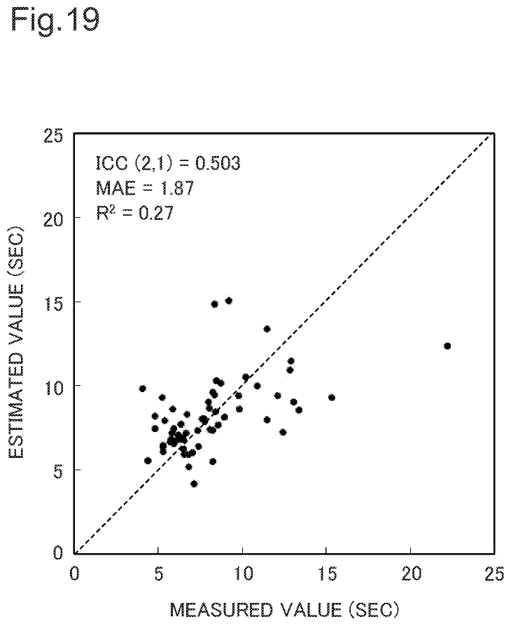

Next, a result of evaluating the estimation model 151 generated using the measurement data of the 62 subjects described above will be described. Here, a verification example (FIG. 18) in which lower limb muscle power (five-time sit-to-stand time) was estimated using the attribute (including walking speed) of the subject is compared with a verification example (FIG. 19) in which lower limb muscle power (five-time sit-to-stand time) was estimated using the feature amount of the gait of the subject. FIGS. 18 and 19 illustrate the results of testing the estimation model generated using the measurement data of 61 people with the measurement data of the remaining one person by means of the leave-one-subject-out (LOSO) method. FIGS. 18 and 19 illustrate the results of performing LOSO on all (62) subjects and associating a predicted value from the test with the measured value (true value). The LOSO test result was evaluated using the values of the intraclass correlation coefficient (ICC), mean absolute error (MAE), and determination coefficient R². As the intraclass correlation coefficient ICC, an intraclass correlation coefficient ICC (2, 1) was used to evaluate inter-examiner reliability.

FIG. 18 illustrates a verification result of an estimation model of a comparative example, which has learned teacher data with sex, age, height, weight, and walking speed as explanatory variables and five-time sit-to-stand time as an objective variable. In the estimation model of the comparative example, the intraclass correlation coefficient ICC (2, 1) was 0.20, the mean absolute error MAE was 2.18, and the determination coefficient R² was 0.06.

FIG. 19 illustrates a verification result of the estimation model 151 of the present example embodiment, which has learned teacher data with the feature amounts F1 to F4 and the age as explanatory variables and the five-time sit-to-stand time as an objective variable. In the estimation model 151 of the present example embodiment, the intraclass correlation coefficient ICC (2, 1) was 0.503, the mean absolute error MAE was 1.87, and the determination coefficient R² was 0.27. That is, the estimation model 151 of the present example embodiment has higher reliability and smaller error than the estimation model of the comparative example, and the objective variable is sufficiently explained by the explanatory variables. In other words, according to the method of the present example embodiment, it is possible to generate the estimation model 151 that is highly reliable, has a small error, and has the objective variable sufficiently explained by the explanatory variables, compared to the estimation model using only the attribute and the walking speed.

### Operation

Next, the operation of the lower limb muscle power estimation system I will be described with Literature to the drawings. Here, the gait measurement device 10 and the lower limb muscle power estimation device 13 included in the lower limb muscle power estimation system 1 will be individually described. With respect to the gait measurement device 10, the operation of the feature amount data generation unit 12 included in the gait measurement device 10 will be described.

### [Gait Measurement Device]

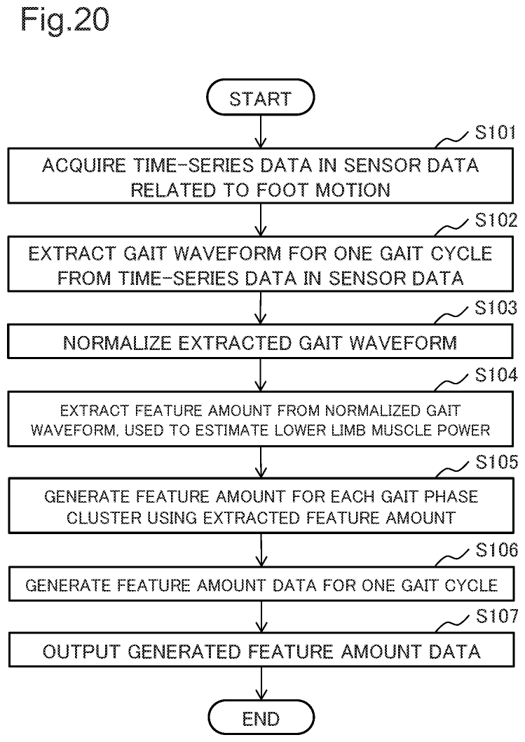

FIG. 20 is a flowchart for explaining the operation of the feature amount data generation unit 12 included in the gait measurement device 10. In the description along the flowchart of FIG. 20, the feature amount data generation unit 12 will be described as the main unit for the operation.

In FIG. 20, first, the feature amount data generation unit 12 acquires time-series data in sensor data related to the foot motion (step S101).

Next, the feature amount data generation unit 12 extracts gait waveform data for one gait cycle from the time-series data in the sensor data (step S102). The feature amount data generation unit 12 detects heel contact and toe-off from the time-series data in the sensor data. The feature amount data generation unit 12 extracts time-series data of a section between consecutive heel contacts as gait waveform data for one gait cycle.

Next, the feature amount data generation unit 12 normalizes the extracted gait waveform data for one gait cycle (step S103). The feature amount data generation unit 12 normalizes the gait waveform data for one gait cycle to a gait cycle of 0 to 100% (first normalization). Further, the feature amount data generation unit 12 normalizes the ratio of the stance phase to the swinging phase in the gait waveform data subjected to the first normalization for one gait cycle to 60:40 (second normalization).

Next, the feature amount data generation unit 12 extracts a feature amount from a gait phase to be used to estimate lower limb muscle power with respect to the normalized gait waveform (step S104). For example, the feature amount data generation unit 12 extracts a feature amount input to an estimation model constructed in advance.

Next, the feature amount data generation unit 12 generates a feature amount for each gait phase cluster using the extracted feature amount (step S105).

Next, the feature amount data generation unit 12 integrates the feature amounts for each gait phase cluster to generate feature amount data for one gait cycle (step S106).

Next, feature amount data generation unit 12 outputs the generated feature amount data to lower limb muscle power estimation device 13 (step S107).

### [Lower Limb Muscle Power Estimation Device]

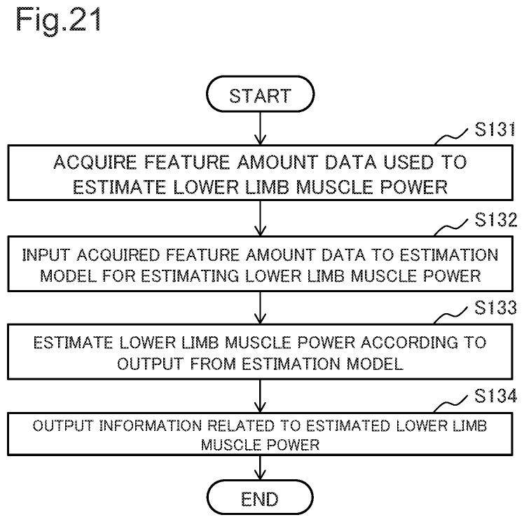

FIG. 21 is a flowchart for explaining the operation of the lower limb muscle power estimation device 13. In the description along the flowchart of FIG. 21, the lower limb muscle power estimation device 13 will be described as the main unit for the operation.

In FIG. 21, first, the lower limb muscle power estimation device 13 acquires feature amount data generated using sensor data related to the foot motion (step S131).

Next, the lower limb muscle power estimation device 13 inputs the acquired feature amount data to an estimation model for estimating lower limb muscle power (five-time sit-to-stand time) (step S132).

Next, the lower limb muscle power estimation device 13 estimates the user's lower limb muscle power according to the output (estimated value) from the estimation model (step S133). For example, the lower limb muscle power estimation device 13 estimates the five-time sit-to-stand time for the user as lower limb muscle power.

Next, the lower limb muscle power estimation device 13 outputs information related to the estimated lower limb muscle power (step S134). For example, the lower limb muscle power is output to a terminal device (not illustrated) carried by the user. For example, the lower limb muscle power is output to a system that executes processing using the lower limb muscle power.

### Application Example

Next, an application example according to the present example embodiment will be described with Literature to the drawings. In the following application example, an example will be shown in which the function of the lower limb muscle power estimation device 13 installed in the mobile terminal carried by the user estimates lower limb muscle power using the feature amount data measured by the gait measurement device placed in the shoe.

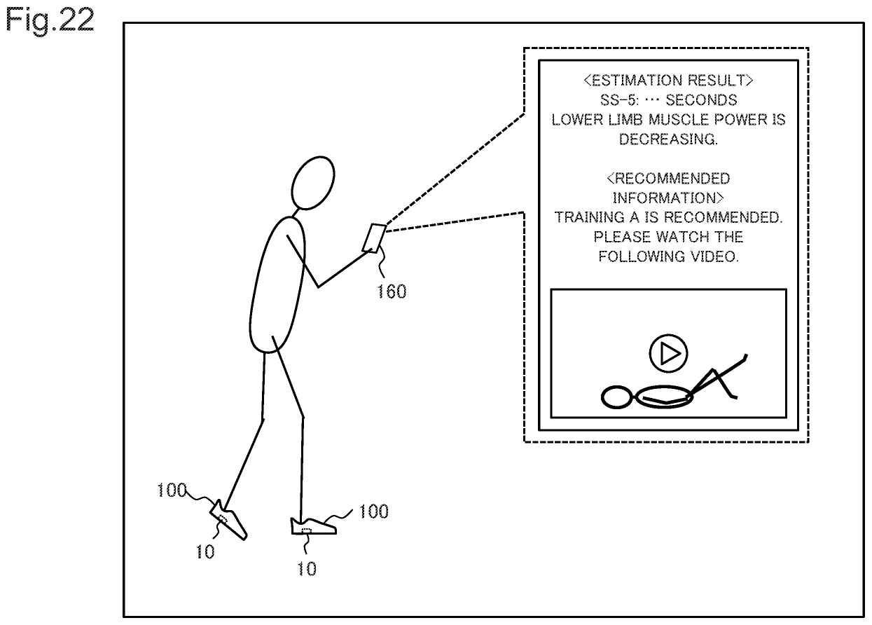

FIG. 22 is a conceptual diagram illustrating an example in which the result of estimation by the lower limb muscle power estimation device 13 is displayed on a screen of a mobile terminal 160 carried by the user walking while wearing the shoe 100 in which the gait measurement device 10 is placed. FIG. 22 is an example in which information corresponding to the estimation result of the lower limb muscle power using the feature amount data corresponding to the sensor data measured while the user is walking is displayed on the screen of the mobile terminal 160.

FIG. 22 illustrates an example in which information corresponding to the estimated value of the five-time sit-to-stand time, which is lower limb muscle power, is displayed on the screen of the mobile terminal 160. In the example of FIG. 22, the estimated value of the five-time sit-to-stand time is displayed on the display unit of the mobile terminal 160 as the estimation result of the lower limb muscle power. In the example of FIG. 22, information related to the estimation result of lower limb muscle power, “LOWER LIMB MUSCLE POWER IS DECREASING”, is displayed on the display unit of the mobile terminal 160 according to the estimated value of the five-time sit-to-stand time, which is the lower limb muscle power. In the example of FIG. 22, recommendation information corresponding to the estimation result of the lower limb muscle power, “TRAINING A IS RECOMMENDED. PLEASE WATCH THE FOLLOWING VIDEO.” is displayed on the display unit of the mobile terminal 160 according to the estimated value of the five-time sit-to-stand time, which is the lower limb muscle power. The user who has confirmed the information displayed on the display unit of the mobile terminal 160 can practice training that leads to an increase in lower limb muscle power by exercising with Literature to the video of training A according to the recommendation information.

As described above, the lower limb muscle power estimation system of the present example embodiment includes the gait measurement device and the lower limb muscle power estimation device. The gait measurement device includes a sensor and a feature amount data generation unit. The sensor includes an acceleration sensor and an angular velocity sensor. The sensor measures a spatial acceleration using an acceleration sensor. The sensor measures a spatial angular velocity using an angular velocity sensor. The sensor generates sensor data related to the foot motion using the measured spatial acceleration and spatial angular velocity. The sensor outputs the generated sensor data to the feature amount data generation unit. The feature amount data generation unit acquires time-series data in the sensor data related to the foot motion. The feature amount data generation unit extracts gait waveform data for one gait cycle from the time-series data in the sensor data. The feature amount data generation unit normalizes the extracted gait waveform data. The feature amount data generation unit extracts a feature amount to be used to estimate lower limb muscle power from a gait phase cluster, formed of at least one temporally continuous gait phase, based on the normalized gait waveform data. The feature amount data generation unit generates feature amount data including the extracted feature amount. The feature amount data generation unit outputs the generated feature amount data.

The lower limb muscle power estimation device includes a data acquisition unit, a storage unit, an estimation unit, and an output unit. The data acquisition unit acquires feature amount data including a feature amount extracted from sensor data related to the user's foot motion and to be used to estimate the user's lower limb muscle power. The storage unit stores an estimation model that outputs a lower limb muscle power index corresponding to the input of the feature amount data. The estimation unit inputs the acquired feature amount data to the estimation model to estimate the user's lower limb muscle power. The output unit outputs information related to the estimated lower limb muscle power.

The lower limb muscle power estimation system of the present example embodiment estimates the user's lower limb muscle power using the feature amount extracted from the sensor data related to the user's foot motion. Therefore, according to the lower limb muscle power estimation system of the present example embodiment, lower limb muscle power can be appropriately estimated in daily life without using an instrument for measuring lower limb muscle power.

In one aspect of the present example embodiment, the data acquisition unit acquires the feature amount data including a feature amount extracted from gait waveform data generated using time-series data in the sensor data related to the foot motion. The data acquisition unit acquires feature amount data including a feature amount to be used to estimate a performance value of the sit-to-stand test as the lower limb muscle power index. According to the present aspect, using the sensor data related to the foot motion makes it possible to appropriately estimate lower limb muscle power in daily life without using an instrument for measuring lower limb muscle power.

In one aspect of the present example embodiment, the storage unit stores an estimation model generated by machine learning using teacher data related to a plurality of subjects. The estimation model is generated by machine learning using teacher data with a feature amount to be used to estimate a lower limb muscle power index as an explanatory variable and the lower limb muscle power index of each of the plurality of subjects as an objective variable. The estimation unit inputs the feature amount data acquired for the user to the estimation model. The estimation unit estimates the user's lower limb muscle power according to the user's lower limb muscle power index output from the estimation model. According to the present aspect, lower limb muscle power can be appropriately estimated in daily life without using an instrument for measuring lower limb muscle power.

In one aspect of the present example embodiment, the storage unit stores the estimation model that has learned using the explanatory variables including the attribute data (age) of the subject. The estimation unit inputs the feature amount data and the attribute data (age) related to the user to the estimation model. The estimation unit estimates the user's lower limb muscle power according to the user's lower limb muscle power index output from the estimation model. In the present aspect, lower limb muscle power is estimated including attribute data (age) that affects lower limb muscle power. Therefore, according to the present aspect, lower limb muscle power can be measured with higher accuracy.

In one aspect of the present example embodiment, the storage unit stores an estimation model generated by machine learning using teacher data related to a plurality of subjects. The estimation model is a model generated by machine learning using teacher data with feature amounts extracted from the gait waveform data of the plurality of subjects as explanatory variables and lower limb muscle power indexes of the plurality of subjects as objective variables. For example, the explanatory variables include a feature amount related to the quadriceps femoris, hamstrings, and gastrocnemius extracted from the early stage of the mid-stance period. For example, the explanatory variables include a feature amount related to the activity of the gastrocnemius extracted from the section from the terminal stance period to the pre-swing period. For example, the explanatory variables include a feature amount related to the activities of the quadriceps femoris, hamstrings, and tibialis anterior extracted from the final stage of the terminal swing period. The estimation unit inputs feature amount data acquired according to the user's gait to the estimation model. The estimation unit estimates the user's lower limb muscle power according to the user's lower limb muscle power index output from the estimation model. According to the present aspect, it is possible to estimate lower limb muscle power more suitable for the physical activity by using the estimation model that has learned a feature amount corresponding to the activity of the muscle that affects the lower limb muscle power.

In one aspect of the present example embodiment, the storage unit stores an estimation model generated by machine learning using teacher data with a plurality of feature amounts extracted from gait waveform data for a plurality of subjects as explanatory variables and lower limb muscle power related to the lower limb muscle power indexes of the subjects as objective variables. For example, the explanatory variables include a feature amount extracted from each of the early stage of the mid-stance period and the final stage of the terminal swing period in the gait waveform data of the angular velocity in the coronal plane. For example, the explanatory variables include a feature amount extracted from a section from the terminal stance period to the pre-swing period of the gait waveform data of the angular velocity in the pre-swing period sagittal plane. For example, the explanatory variables include a feature amount extracted from the final stage of the terminal swing period of the angle in the horizontal plane. The data acquisition unit acquires feature amount data including a feature amount extracted according to the user's gait. For example, the data acquisition unit acquires a feature amount at each of the early stage of the mid-stance period and the final stage of the terminal swing period of the gait waveform data of the angular velocity in the coronal plane. For example, the data acquisition unit acquires a feature amount for a section from the terminal stance period to the pre-swing period of the gait waveform data of the angular velocity in the sagittal plane. For example, the data acquisition unit acquires a feature amount at the final stage of the terminal swing period of the gait waveform data at an angle in the horizontal plane. The estimation unit inputs the acquired feature amount data to the estimation model. The estimation unit estimates the user's lower limb muscle power according to the user's lower limb muscle power index output from the estimation model. According to the present aspect, the lower limb muscle power more suitable for the physical activity can be estimated using the sensor data related to the foot motion by using the estimation model that has learned the feature amount extracted from the gait waveform data including the feature corresponding to the activity of the muscle that affects the lower limb muscle power.

In one aspect of the present example embodiment, the lower limb muscle power estimation device is mounted on a terminal device including a screen that can be viewed by the user. For example, the lower limb muscle power estimation device causes the screen of the terminal device to display information related to the lower limb muscle power estimated according to the user's foot motion. For example, the lower limb muscle power estimation device causes the screen of the terminal device to display recommendation information corresponding to the lower limb muscle power estimated according to the user's foot motion. For example, the lower limb muscle power estimation device causes the screen of the terminal device to display a video related to training for training a body site related to lower limb muscle power as the recommendation information corresponding to the lower limb muscle power estimated according to the user's foot motion. According to the present aspect, the lower limb muscle power estimated according to the feature amount extracted from the sensor data related to the user's foot motion is displayed on the screen that can be viewed by the user, so that the user can confirm the information corresponding to the user's lower limb muscle power.

### Second Example Embodiment

Next, a machine learning system according to a second example embodiment will be described with Literature to the drawings. The machine learning system of the present example embodiment generates an estimation model for estimating lower limb muscle power according to input of a feature amount by machine learning using feature amount data extracted from sensor data measured by a gait measurement device.

### (Configuration)

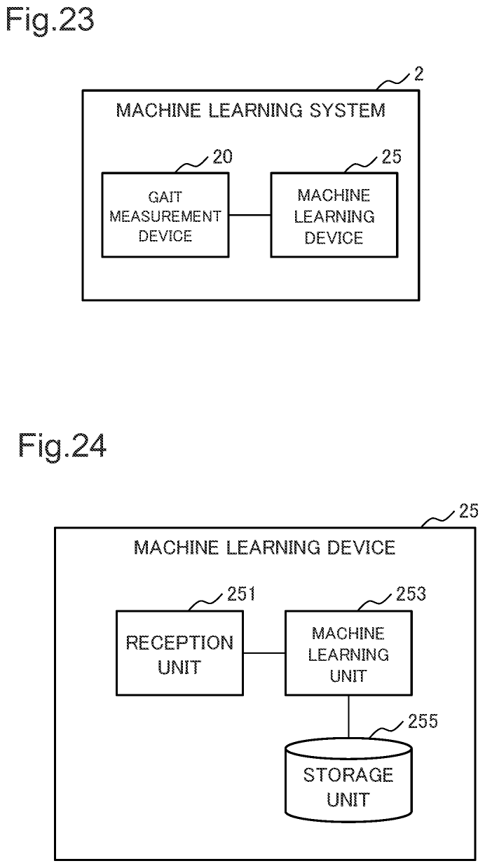

FIG. 23 is a block diagram illustrating an example of the configuration of a machine learning system 2 according to the present example embodiment. The machine learning system 2 includes a gait measurement device 20 and a machine learning device 25. The gait measurement device 20 and the machine learning device 25 may be connected by wire or wirelessly. The gait measurement device 20 and the machine learning device 25 may be configured using a single device. The machine learning system 2 may be formed only of the machine learning device 25, with the gait measurement device 20 excluded from the configuration of the machine learning system 2. Although only one gait measurement device 20 is illustrated in FIG. 23, one gait measurement device 20 may be placed on each of the left and right feet (two devices in total). The machine learning device 25 may be configured not to be connected to the gait measurement device 20 but to execute machine learning using the feature amount data generated in advance by the gait measurement device 20 and stored in the database.

The gait measurement device 20 is fitted to at least one of the left and right feet. The gait measurement device 20 has a similar configuration to that of the gait measurement device 10 of the first example embodiment. The gait measurement device 20 includes an acceleration sensor and an angular velocity sensor. The gait measurement device 20 converts the measured physical quantity into digital data (also referred to as sensor data). The gait measurement device 20 generates normalized gait waveform data for one gait cycle from the time-series data in the sensor data. The gait measurement device 20 generates feature amount data to be used to estimate lower limb muscle power, which is an estimation target. The gait measurement device 20 transmits the generated feature amount data to the machine learning device 25. The gait measurement device 20 may be configured to transmit the feature amount data to a database (not illustrated) accessed by the machine learning device 25. The feature amount data accumulated in the database is used for machine learning by the machine learning device 25.

The machine learning device 25 receives the feature amount data from the gait measurement device 20. In a case where the feature amount data accumulated in the database (not illustrated) is used, the machine learning device 25 receives the feature amount data from the database. The machine learning device 25 executes machine learning using the received feature amount data. For example, the machine learning device 25 learns teacher data with feature amount data extracted from gait waveform data of a plurality of subjects as an explanatory variable and a value related to lower limb muscle power corresponding to the feature amount data as an objective variable. The machine learning algorithm executed by the machine learning device 25 is not particularly limited. The machine learning device 25 generates an estimation model that has learned using teacher data related to the plurality of subjects. The machine learning device 25 stores the generated estimation model. The estimation model trained by the machine learning device 25 may be stored in a storage device outside the machine learning device 25.

### [Machine Learning Device]

Next, details of the machine learning device 25 will be described with Literature to the drawings. FIG. 24 is a block diagram illustrating an example of a detailed configuration of the machine learning device 25. The machine learning device 25 includes a reception unit 251, a machine learning unit 253, and a storage unit 255.

The reception unit 251 receives feature amount data from the gait measurement device 20. The reception unit 251 outputs the received feature amount data to the machine learning unit 253. The reception unit 251 may receive the feature amount data from the gait measurement device 20 via a wire such as a cable, or may receive the feature amount data from the gait measurement device 20 via wireless communication. For example, the reception unit 251 is configured to receive the feature amount data from the gait measurement device 20 via a wireless communication function (not illustrated) conforming to a standard such as Bluetooth (registered trademark) or WiFi (registered trademark). The communication function of the reception unit 251 may conform to a standard other than Bluetooth (registered trademark) or WiFi (registered trademark).

The machine learning unit 253 acquires the feature amount data from the reception unit 251. The machine learning unit 253 executes machine learning using the acquired feature amount data. For example, the machine learning unit 253 learns, as teacher data, a dataset in which feature amount data extracted from the sensor data measured according to the foot motion of the subject is used as an explanatory variable, and the five-time sit-to-stand time of the subject is used as an objective variable. For example, the machine learning unit 253 generates an estimation model that has learned for the plurality of subjects to estimate the five-time sit-to-stand time according to the input of the feature amount data. For example, the machine learning unit 253 generates an estimation model corresponding to attribute data (age). For example, the machine learning unit 253 generates an estimation model that estimates the five-time sit-to-stand time as lower limb muscle power, using the feature amount data extracted from the sensor data measured according to the foot motion of the subject and the attribute data (age) of the subject as explanatory variables. The machine learning unit 253 stores the estimation model that has learned for the plurality of subjects in the storage unit 255.

For example, the machine learning unit 253 executes machine learning using a linear regression algorithm. For example, the machine learning unit 253 executes machine learning using an algorithm of a support-vector machine (SVM). For example, the machine learning unit 253 executes machine learning using a gaussian process regression (GPR) algorithm. For example, the machine learning unit 253 executes machine learning using a random forest (RF) algorithm. For example, the machine learning unit 253 may execute unsupervised machine learning of classifying a subject who is a generation source of the feature amount data according to the feature amount data. The machine learning algorithm executed by the machine learning unit 253 is not particularly limited.

The machine learning unit 253 may execute machine learning using the gait waveform data for one gait cycle as an explanatory variable. For example, the machine learning unit 253 executes supervised machine learning using the gait waveform data of the accelerations in the three axial directions, the angular velocities around the three axes, and the angles (posture angles) around the three axes as explanatory variables, and using the correct value of the lower limb muscle power to be estimated as an objective variable. For example, when the gait phase has been set in increments of 1% in the gait cycle of 0 to 100%, the machine learning unit 253 learns using 909 explanatory variables.

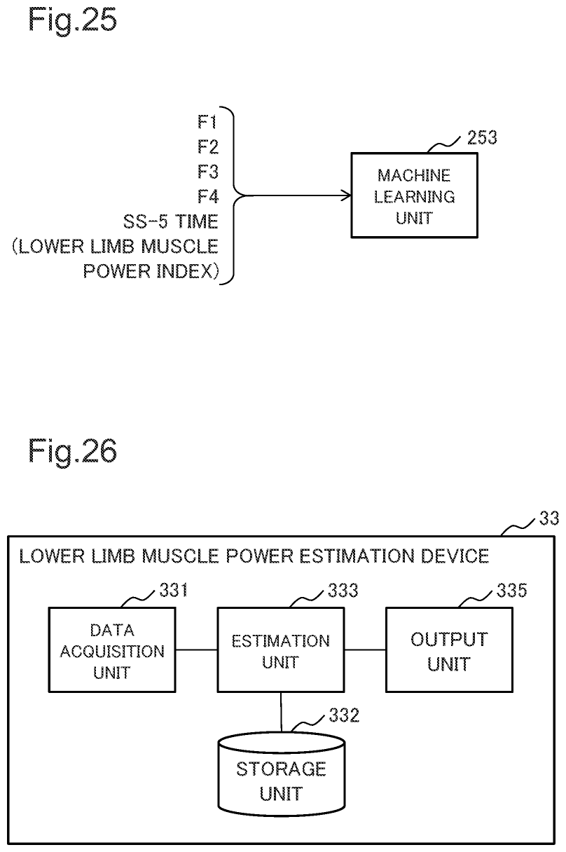

FIG. 25 is a conceptual diagram for explaining machine learning for generating an estimation model. FIG. 25 is a conceptual diagram illustrating an example in which the machine learning unit 253 is caused to learn a dataset of the feature amounts F1 to F4 as explanatory variables and the five-time sit-to-stand time (lower limb muscle power index) as an objective variable as teacher data. For example, the machine learning unit 253 learns data related to a plurality of subjects, and generates an estimation model that outputs an output (estimated value) related to the five-time sit-to-stand time (lower limb muscle power index) according to an input of a feature amount extracted from the sensor data.

The storage unit 255 stores the estimation model that has learned for each of the plurality of subjects. For example, the storage unit 255 stores an estimation model that has learned for the plurality of subjects to estimate lower limb muscle power. For example, the estimation model stored in the storage unit 255 is to be used to estimate lower limb muscle power by the lower limb muscle power estimation device 13 of the first example embodiment.

As described above, the machine learning system of the present example embodiment includes the gait measurement device and the machine learning device. The gait measurement device acquires time-series data in sensor data related to foot motion. The gait measurement device extracts gait waveform data for one gait cycle from the time-series data in the sensor data, and normalizes the extracted gait waveform data. The gait measurement device extracts a feature amount to be used to estimate the user's lower limb muscle power from a gait phase cluster, formed of at least one temporally continuous gait phase, based on the normalized gait waveform data. The gait measurement device generates feature amount data including the extracted feature amount. The gait measurement device outputs the generated feature amount data to the machine learning device.

The machine learning device includes a reception unit, a machine learning unit, and a storage unit. The reception unit acquires the feature amount data generated by the gait measurement device. The machine learning unit executes machine learning using the feature amount data. The machine learning unit generates the estimation model that outputs the lower limb muscle power according to the input of the feature amount (second feature amount) of the gait phase cluster extracted from the time-series data in the sensor data measured along with the user's gait. The estimation model generated by the machine learning unit is stored in the storage unit.

The machine learning system of the present example embodiment generates an estimation model, using the feature amount data measured by the gait measurement device. Therefore, according to the present aspect, it is possible to generate an estimation model capable of appropriately estimating lower limb muscle power in daily life without using an instrument for measuring lower limb muscle power.

### Third Example Embodiment

Next, a lower limb muscle power estimation device according to a third example embodiment will be described with Literature to the drawings. The lower limb muscle power estimation device of the present example embodiment has a simplified configuration of the lower limb muscle power estimation device included in the lower limb muscle power estimation system of the first example embodiment.

FIG. 26 is a block diagram illustrating an example of a configuration of a lower limb muscle power estimation device 33 according to the present example embodiment. The lower limb muscle power estimation device 33 includes a data acquisition unit 331, a storage unit 332, an estimation unit 333, and an output unit 335.

The data acquisition unit 331 acquires feature amount data including a feature amount extracted from sensor data related to the user's foot motion and to be used to estimate the user's lower limb muscle power index. The storage unit 332 stores an estimation model that outputs the lower limb muscle power index corresponding to the input of the feature amount data. The estimation unit 333 inputs the acquired feature amount data to the estimation model, and estimates the user's lower limb muscle power according to the lower limb muscle power index output from the estimation model. The output unit 335 outputs information related to the estimated lower limb muscle power.

As described above, in the present example embodiment, the user's lower limb muscle power is estimated using the feature amount extracted from the sensor data related to the user's foot motion. Therefore, according to the present example embodiment, lower limb muscle power can be appropriately estimated in daily life without using an instrument for measuring lower limb muscle power.

### [Hardware Configuration]

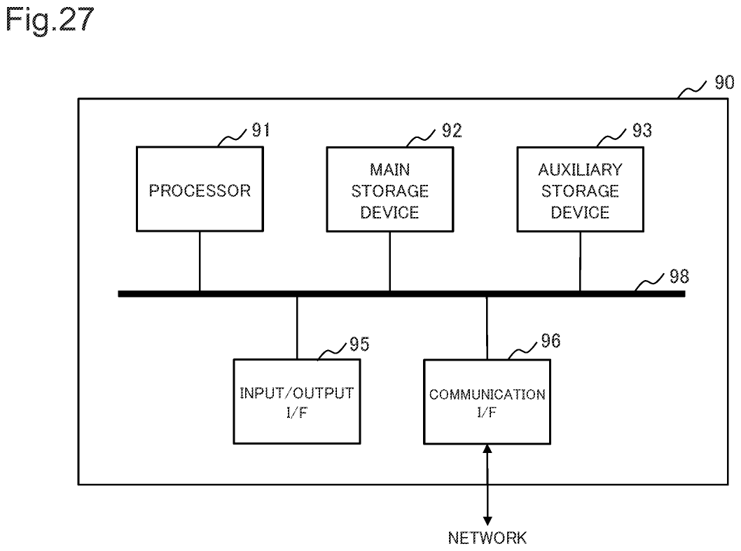

Here, a hardware configuration for executing the control and processing according to each example embodiment of the present disclosure will be described using an information processing device 90 of FIG. 27 as an example. The information processing device 90 in FIG. 27 is a configuration example for executing the control and processing of each example embodiment and does not limit the scope of the present disclosure.

As illustrated in FIG. 27, the information processing device 90 includes a processor 91, a main storage device 92, an auxiliary storage device 93, an input/output interface 95, and a communication interface 96. In FIG. 27, the interface is abbreviated as I/F. The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program stored in the auxiliary storage device 93 or the like in the main storage device 92. The processor 91 executes the program developed in the main storage device 92. In the present example embodiment, a software program installed in the information processing device 90 may be used. The processor 91 executes the control and processing according to each example embodiment.

The main storage device 92 has an area in which a program is developed. A program stored in the auxiliary storage device 93 or the like is developed in the main storage device 92 by the processor 91. The main storage device 92 is achieved by, for example, a volatile memory such as a dynamic random-access memory (DRAM). A nonvolatile memory such as a magnetoresistive random-access memory (MRAM) may be configured/added as the main storage device 92.

The auxiliary storage device 93 stores various data such as programs. The auxiliary storage device 93 is achieved by a local disk such as a hard disk or a flash memory. Various data may be stored in the main storage device 92, and the auxiliary storage device 93 may be omitted.

The input/output interface 95 is an interface for connecting the information processing device 90 and a peripheral device, based on a standard or a specification. The communication interface 96 is an interface for connecting to an external system or device through a network such as the Internet or an intranet, based on a standard or a specification. The input/output interface 95 and the communication interface 96 may be shared as an interface connected to an external device.

Input devices such as a keyboard, a mouse, and a touch panel may be connected to the information processing device 90 as necessary. These input devices are used to input information and settings. When the touch panel is used as the input device, the display screen of the display device may also serve as the interface of the input device. Data communication between the processor 91 and the input device may be mediated by the input/output interface 95.

The information processing device 90 may be equipped with a display device for displaying information. In a case where a display device is provided, the information processing device 90 preferably includes a display control device (not illustrated) for controlling the display of the display device. The display device may be connected to the information processing device 90 via the input/output interface 95.

The information processing device 90 may be equipped with a drive device. The drive device mediates reading of data and a program from a recording medium (program recording medium), writing of a processing result of the information processing device 90 to the recording medium, and the like between the processor 91 and the recording medium. The drive device may be connected to the information processing device 90 via the input/output interface 95.

The above is an example of a hardware configuration for enabling the control and processing according to each example embodiment of the present invention. The hardware configuration of FIG. 27 is an example of a hardware configuration for executing the control and processing according to each example embodiment, and does not limit the scope of the present invention. A program for causing a computer to execute the control and processing according to each example embodiment is also included in the scope of the present invention. Further, a program recording medium in which the program according to each example embodiment is recorded is also included in the scope of the present invention. The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card. The recording medium may be achieved by a magnetic recording medium such as a flexible disk, or another recording medium. When a program executed by the processor is recorded in a recording medium, the recording medium corresponds to a program recording medium.

The components of each example embodiment may be combined in any manner. The components of each example embodiment may be achieved by software or may be achieved by a circuit.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

Some or all of the above example embodiments may be described as the following Supplementary Notes, but are not limited to the following.

### Supplementary Note 1

A lower limb muscle power estimation device including:
a data acquisition unit that acquires feature amount data including a feature amount extracted from sensor data related to foot motion of a user, the feature amount being used to estimate lower limb muscle power of the user;
a storage unit that stores an estimation model for outputting a lower limb muscle power index corresponding to input of the feature amount data;
an estimation unit that inputs the acquired feature amount data to the estimation model and estimate the lower limb muscle power of the user according to the lower limb muscle power index output from the estimation model; and
an output unit that outputs information related to the estimated lower limb muscle power of the user.

### Supplementary Note 2

The lower limb muscle power estimation device according to Supplementary Note 1, in which
the data acquisition unit acquires the feature amount data including a feature amount extracted from gait waveform data generated using time-series data in the sensor data related to the foot motion, the feature amount being used to estimate a performance value of a sit-to-stand test as the lower limb muscle power index.

### Supplementary Note 3

The lower limb muscle power estimation device according to Supplementary Note 2, in which
the storage unit stores the estimation model generated by machine learning using teacher data with a feature amount used to estimate the lower limb muscle power index for each of a plurality of subjects as an explanatory variable and the lower limb muscle power index of each of the plurality of subjects as an objective variable, and
the estimation unit inputs the feature amount data acquired for the user to the estimation model, and estimates the lower limb muscle power of the user according to the lower limb muscle power index of the user output from the estimation model.

### Supplementary Note 4

The lower limb muscle power estimation device according to Supplementary Note 3, in which
the storage unit stores the estimation model that has learned using explanatory variables including an age of each of the plurality of subjects, and
the estimation unit inputs the feature amount data and an age related to the user to the estimation model, and estimates the lower limb muscle power of the user according to the lower limb muscle power index of the user output from the estimation model.

### Supplementary Note 5

The lower limb muscle power estimation device according to Supplementary Note 3 or 4, in which
the storage unit stores the estimation model generated by machine learning using teacher data having, for the gait waveform data of each of the plurality of subjects, a feature amount related to the quadriceps femoris, hamstrings, and gastrocnemius extracted from an early stage of a mid-stance period, a feature amount related to an activity of the gastrocnemius extracted from a section from a terminal stance period to a pre-swing period, and a feature amount related to activities of the quadriceps femoris, hamstrings, and tibialis anterior extracted from a final stage of a terminal swing period as explanatory variables, the teacher data having the lower limb muscle power index of each of the plurality of subjects as an objective variable, and
the estimation unit inputs the feature amount data acquired according to gait of the user to the estimation model, and estimates the lower limb muscle power of the user according to the lower limb muscle power index of the user output from the estimation model.

### Supplementary Note 6

The lower limb muscle power estimation device according to Supplementary Note 5, in which
the storage unit stores the estimation model generated by machine learning using teacher data having, for each of the plurality of subjects, a feature amount extracted from each of an early stage of a mid-stance period and a final stage of a terminal swing period of the gait waveform data of an angular velocity in a coronal plane, a feature amount extracted from a section from a terminal stance period to a pre-swing period of the gait waveform data of an angular velocity in a sagittal plane, and a feature amount extracted from a final stage of a terminal swing period of the gait waveform data of an angle in a horizontal plane as explanatory variables, the teacher data using the lower limb muscle power index of each of the plurality of subjects as an objective variable,
the data acquisition unit acquires the feature amount data including the feature amount at each of the early stage of the mid-stance period and the final stage of the terminal swing period of the gait waveform data of the angular velocity in the coronal plane, the feature amount for the section from the terminal stance period to the pre-swing period of the gait waveform data of the angular velocity in the sagittal plane, and the feature amount at the final stage of the terminal swing period of the gait waveform data of the angle in the horizontal plane, each of the feature amounts having been extracted according to gait of the user, and
the estimation unit inputs the acquired feature amount data to the estimation model, and estimates the lower limb muscle power of the user according to the lower limb muscle power index of the user output from the estimation model.

### Supplementary Note 7

The lower limb muscle power estimation device according to any one of Supplementary Notes 3 to 6, in which
the estimation unit estimates information related to the lower limb muscle power of the user according to the lower limb muscle power index estimated for the user, and
the output unit outputs the estimated information related to the lower limb muscle power.

### Supplementary Note 8

A lower limb muscle power estimation system including:
the lower limb muscle power estimation device according to any one of Supplementary Notes 1 to 7; and
a gait measurement device including a sensor that is fitted to footwear of a user who is an estimation target for lower limb muscle power, measures a spatial acceleration and a spatial angular velocity, generates sensor data related to foot motion using the measured spatial acceleration and the measured spatial angular velocity, and outputs the generated sensor data, the gait measurement device including a feature amount data generation unit that acquires time-series data in the sensor data including a feature of gait, extracts gait waveform data for one gait cycle from the time-series data in the sensor data, normalizes the extracted gait waveform data, extracts a feature amount to be used to estimate the lower limb muscle power from a gait phase cluster, including at least one temporally continuous gait phase, based on the normalized gait waveform data, generates feature amount data including the extracted feature amount, and outputs the generated feature amount data to the lower limb muscle power estimation device.

### Supplementary Note 9

The lower limb muscle power estimation system according to Supplementary Note 8, in which the lower limb muscle power estimation device
is implemented in a terminal device including a screen that can be viewed by the user, and
causes the screen of the terminal device to display information related to the lower limb muscle power estimated according to the foot motion of the user.

### Supplementary Note 10

The lower limb muscle power estimation system according to Supplementary Note 9, in which
the lower limb muscle power estimation device causes the screen of the terminal device to display recommendation information corresponding to the lower limb muscle power estimated according to the foot motion of the user.

### Supplementary Note 11

The lower limb muscle power estimation system according to Supplementary Note 10, in which
the lower limb muscle power estimation device causes the screen of the terminal device to display a video related to training for training a body site related to lower limb muscle power as the recommendation information corresponding to the lower limb muscle power estimated according to the foot motion of the user.

### Supplementary Note 12

A lower limb muscle power estimation method executed by a computer, the method including:
acquiring feature amount data that includes a feature amount extracted from sensor data related to foot motion of a user, the feature amount being used to estimate lower limb muscle power of the user;
inputting the acquired feature amount data to an estimation model for outputting a lower limb muscle power index corresponding to input of the feature amount data;
estimating the lower limb muscle power of the user according to the lower limb muscle power index output from the estimation model; and outputting information related to the estimated lower limb muscle power of the user.

### Supplementary Note 13

A program that causes a computer to execute
acquiring feature amount data that includes a feature amount extracted from sensor data related to foot motion of a user, the feature amount being used to estimate lower limb muscle power of the user,
inputting the acquired feature amount data to an estimation model for outputting a lower limb muscle power index corresponding to input of the feature amount data,
estimating the lower limb muscle power of the user according to the lower limb muscle power index output from the estimation model, and
outputting information related to the estimated lower limb muscle power of the user.

### REFERENCE SIGNS LIST

1 lower limb muscle power estimation system
2 machine learning system
10, 20 gait measurement device
11 sensor
12 feature amount data generation unit
13 lower limb muscle power estimation device
25 machine learning device
111 acceleration sensor
112 angular velocity sensor
121 acquisition unit
122 normalization unit
123 extraction unit
125 generation unit
127 feature amount data output unit
131, 331 data acquisition unit
132, 332 storage unit
133, 333 estimation unit
135, 335 output unit
251 reception unit
253 machine learning unit
255 storage unit

What is claimed is:

1. A lower limb muscle power estimation device comprising:
a storage configured to store an estimation model that outputs a lower limb muscle power index corresponding to input of feature amount data used for estimating lower limb muscle power;
a memory storing instructions; and
a processor connected to the memory and configured to execute the instructions to:
acquire feature amount data including a feature amount extracted from sensor data related to foot motion of a user, the feature amount being used to estimate lower limb muscle power of the user;
input the acquired feature amount data to the estimation model and estimate the lower limb muscle power of the user according to the lower limb muscle power index output from the estimation model; and
output information related to the estimated lower limb muscle power of the user.

2. The lower limb muscle power estimation device according to claim 1, wherein
the processor is configured to execute the instructions to acquire the feature amount data including a feature amount extracted from gait waveform data generated using time-series data in the sensor data related to the foot motion, the feature amount being used to estimate a performance value of a sit-to-stand test as the lower limb muscle power index.

3. The lower limb muscle power estimation device according to claim 2, wherein
the storage stores the estimation model generated by machine learning using teacher data with a feature amount used to estimate the lower limb muscle power index for each of a plurality of subjects as an explanatory variable and the lower limb muscle power index of each of the plurality of subjects as an objective variable, and
the processor is configured to execute the instructions to input the feature amount data acquired for the user to the estimation model, and estimate the lower limb muscle power of the user according to the lower limb muscle power index of the user output from the estimation model.

4. The lower limb muscle power estimation device according to claim 3, wherein
the storage stores the estimation model that has learned using explanatory variables including an age of each of the plurality of subjects, and
the processor is configured to execute the instructions to
input the feature amount data and an age related to the user to the estimation model, and
estimate the lower limb muscle power of the user according to the lower limb muscle power index of the user output from the estimation model.

5. The lower limb muscle power estimation device according to claim 3, wherein
the storage stores the estimation model generated by machine learning using teacher data having, for the gait waveform data of each of the plurality of subjects, a feature amount related to the quadriceps femoris, hamstrings, and gastrocnemius extracted from an early stage of a mid-stance period, a feature amount related to an activity of the gastrocnemius extracted from a section from a terminal stance period to a pre-swing period, and a feature amount related to activities of the quadriceps femoris, hamstrings, and tibialis anterior extracted from a final stage of a terminal swing period as explanatory variables, the teacher data having the lower limb muscle power index of each of the plurality of subjects as an objective variable, and
the processor is configured to execute the instructions to
input the feature amount data acquired according to gait of the user to the estimation model, and
estimate the lower limb muscle power of the user according to the lower limb muscle power index of the user output from the estimation model.

6. The lower limb muscle power estimation device according to claim 5, wherein
the storage stores the estimation model generated by machine learning using teacher data having, for each of the plurality of subjects, a feature amount extracted from each of an early stage of a mid-stance period and a final stage of a terminal swing period of the gait waveform data of an angular velocity in a coronal plane, a feature amount extracted from a section from a terminal stance period to a pre-swing period of the gait waveform data of an angular velocity in a sagittal plane, and a feature amount extracted from a final stage of a terminal swing period of the gait waveform data of an angle in a horizontal plane as explanatory variables, the teacher data using the lower limb muscle power index of each of the plurality of subjects as an objective variable,
the processor is configured to execute the instructions to
acquire the feature amount data including the feature amount at each of the early stage of the mid-stance period and the final stage of the terminal swing period of the gait waveform data of the angular velocity in the coronal plane, the feature amount for the section from the terminal stance period to the pre-swing period of the gait waveform data of the angular velocity in the sagittal plane, and the feature amount at the final stage of the terminal swing period of the gait waveform data of the angle in the horizontal plane, each of the feature amounts having been extracted according to gait of the user,
input the acquired feature amount data to the estimation model, and
estimate the lower limb muscle power of the user according to the lower limb muscle power index of the user output from the estimation model.

7. The lower limb muscle power estimation device according to claim 3, wherein
the processor is configured to execute the instructions to
estimate information related to the lower limb muscle power of the user according to the lower limb muscle power index estimated for the user, and
output the estimated information related to the lower limb muscle power.

8. A lower limb muscle power estimation system comprising:
the lower limb muscle power estimation device according to claim 1; and
a gait measurement device including:
a sensor that is fitted to footwear of a user who is an estimation target for lower limb muscle power, the sensor measures a spatial acceleration and a spatial angular velocity, generates sensor data related to foot motion using the measured spatial acceleration and the measured spatial angular velocity, and output the generated sensor data; and
a memory storing instructions; and
a processor connected to the memory and configured to execute the instructions to:
acquire time-series data in the sensor data including a feature of gait,
extract gait waveform data for one gait cycle from the time-series data in the sensor data,
normalize the extracted gait waveform data,
extract a feature amount to be used to estimate the lower limb muscle power from a gait phase cluster, including at least one temporally continuous gait phase, based on the normalized gait waveform data,
generate feature amount data including the extracted feature amount, and
output the generated feature amount data to the lower limb muscle power estimation device.

9. The lower limb muscle power estimation system according to claim 8, wherein
the lower limb muscle power estimation device is implemented in a terminal device including a screen that can be viewed by the user, and
the processer of the lower limb muscle power estimation device is configured to execute the instructions to cause the screen of the terminal device to display information related to the lower limb muscle power estimated according to the foot motion of the user.

10. The lower limb muscle power estimation system according to claim 9, wherein
the processer of the lower limb muscle power estimation device is configured to execute the instructions to cause the screen of the terminal device to display recommendation information corresponding to the lower limb muscle power estimated according to the foot motion of the user.

11. The lower limb muscle power estimation system according to claim 10, wherein
the processer of the lower limb muscle power estimation device is configured to execute the instructions to cause the screen of the terminal device to display a video related to training for training a body site related to lower limb muscle power as the recommendation information corresponding to the lower limb muscle power estimated according to the foot motion of the user.

12. A lower limb muscle power estimation method executed by a computer, the method comprising:
acquiring feature amount data that includes a feature amount extracted from sensor data related to foot motion of a user, the feature amount being used to estimate lower limb muscle power of the user;
inputting the acquired feature amount data to an estimation model for outputting a lower limb muscle power index corresponding to input of the feature amount data;
estimating the lower limb muscle power of the user according to the lower limb muscle power index output from the estimation model; and
outputting information related to the estimated lower limb muscle power of the user.

13. A non-transitory recording medium recording a program that causes a computer to execute:
processing of acquiring feature amount data that includes a feature amount extracted from sensor data related to foot motion of a user, the feature amount being used to estimate lower limb muscle power of the user,
processing of inputting the acquired feature amount data to an estimation model for outputting a lower limb muscle power index corresponding to input of the feature amount data,
processing of estimating the lower limb muscle power of the user according to the lower limb muscle power index output from the estimation model, and
processing of outputting information related to the estimated lower limb muscle power of the user.

14. The lower limb muscle power estimation system according to claim 10, wherein
the processor of the lower limb muscle power estimation device is configured to execute the instructions to
cause the recommendation information that supports the user for making decision about taking an action to be displayed on the screen of the terminal device.
